(19) United States
(12) Patent Application Publication
(10) Pub. No.: US 2021/0327535 A1
(43) Pub. Date: Oct. 21, 2021

(54) SENSITIVELY DETECTING COPY NUMBER VARIATIONS (CNVS) FROM CIRCULATING CELL-FREE NUCLEIC ACID

(71) Applicants: The Regents of the University of California, Oakland, CA (US); EarlyDiagnostics, Inc., Los Angeles, CA (US)

(72) Inventors: Xianghong Jasmine ZHOU, Los Angeles, CA (US); Wenyuan LI, Los Angeles, CA (US); Shuo LI, Los Angeles, CA (US); Chun-Chi LIU, Los Angeles, CA (US); Xiaohui NI, Los Angeles, CA (US)

(21) Appl. No.: 17/269,983

(22) PCT Filed: Aug. 22, 2019
(86) PCT No.: PCT/US2019/047741

§ 371 Date: Feb. 19, 2021

Related U.S. Application Data
(60) Provisional application No. 62/721,410, filed on Aug. 22, 2018.

(51) Int. Cl. G16B 20/10; G16B 30/00; G16B 40/30

(52) U.S. Cl. CPC G16B 20/10; G16B 40/30; G16B 30/00

(57) ABSTRACT

The present disclosure provides methods and systems for detecting or inferring levels of Copy Number Variants (CNVs) in cell-free nucleic acid samples to detect or assess cancer and prenatal diseases. Cell-free nucleic acid methylation sequencing data may be utilized to distinguish tumor-derived or fetal-derived sequencing reads from normal cfDNA sequencing reads. Each cell-free nucleic acid sequencing read (e.g., containing tumor or fetal methylation markers) may be classified as corresponding to a tumor/fetal-derived or a normal-plasma cell-free nucleic acid, based on the methylation cfDNA sequencing data (e.g., obtained using Bisulfite sequencing or bisulfite-free sequencing methods) and tumor/fetal methylation markers. Next, a profile of the tumor/fetal-derived sequencing read counts may be constructed and then normalized. The CNV status (e.g., gain or loss) of each genomic region may be inferred, and a diagnosis or prognosis can be made based on a subjects inferred CNV profile.

### CROSS-REFERENCE TO RELATED APPLICATIONS

This application claims the benefit of U.S. Provisional Patent Application No. 62/721,410, filed Aug. 22, 2018, which is incorporated by reference herein in its entirety.

### GOVERNMENT INTEREST

This invention was made with Government support under HL108645, awarded by the National Institutes of Health. The Government has certain rights in the invention.

### BACKGROUND

Circulating cell-free nucleic acids, such as cell-free DNA (cfDNA) and cell-free RNA (cfRNA) (e.g., found in plasma), are regarded as a biomarkers of great potential in cancer and prenatal diagnosis and prognosis. As such, the detection and characterization of cfDNA and/or cfRNA represent a promising approach to cancer and prenatal diagnosis and prognosis. Further, because cfDNA and/or cfRNA analysis involves performing a liquid biopsy, rather than a traditional tissue biopsy, it allows for diagnosis, prognosis, or other assessment of a variety of different malignancies without requiring invasive procedures.

Copy number variations, copy number alterations, copy number aberrations, or copy number polymorphisms (collectively referred to as Copy Number Variants (CNVs)) are structurally variant regions in which copy number differences are observed between two or more genomes. Somatic CNVs have critical roles in the development of human cancers through the amplification of oncogenes and deletion of tumor suppressors. Therefore, detecting CNVs from cfDNA and/or cfRNA may provide an effective cancer and prenatal diagnosis and prognosis mechanism.

Typically, a sample of cfDNA obtained from cancer patients comprises a mixture of DNA originating from tumor cells and DNA originating from normal (e.g., non-tumor) cells. Likewise, a sample of cfRNA obtained from cancer patients comprises a mixture of RNA originating from tumor cells and RNA originating from normal (e.g., non-tumor) cells. The challenge in detecting CNVs from cfDNA and/or cfRNA may be exacerbated when there is a low fraction of tumor-derived cfDNA and/or cfRNA in the blood stream. This low fraction of tumor-derived cell-free nucleic acids may make it particularly difficult to differentiate actual variations (e.g., somatic variants such as CNVs) from errors in observation or measurement (e.g., arising from amplification or sequencing errors).

CNVs can be detected by utilizing sequencing-based methods such as Paired-End Mapping (PEM), Split Reads (SR), de novo Assembly (AS), and/or Read-Counts (RC) methods. PEM, SR, and AS methods may comprise searching for discordant sequencing reads or read-pairs that span CNV breakpoints. However, these methods may be impractical for detecting CNVs from cfDNA/cfRNA samples, e.g., where the number of tumor-derived cfDNA/cfRNA sequencing reads is typically very limited, and the chances of identifying discordant reads that exactly span CNV breakpoints are low. Thus, only RC methods, which examine an increase or decrease in the number of sequencing reads within a set of genomic regions, may be practically utilized for CNV detection in cfDNA/cfRNA samples. However, the usefulness of RC methods decreases when the tumor-derived cfDNA fraction in a sample is low. This is because the signal from sequencing reads having tumor CNVs is overwhelmed by the signal from non-tumor sequencing reads, which represent the vast majority of the sample.

### SUMMARY

In view of the foregoing, the present disclosure provides a system and method for detecting or inferring levels of Copy Number Variants (CNVs) in cell-free nucleic acid samples, such as in cases where an amount or level of CNVs in a cell-free nucleic acid sample is low. First, the cfDNA/cfRNA methylation sequencing data and cancer methylation markers may be utilized to distinguish tumor-derived sequencing reads from normal sequencing reads. Each cfDNA/cfRNA sequencing read among a plurality of cfDNA/cfRNA sequencing reads (e.g., containing cancer methylation markers) may be classified as either a tumor-derived cfDNA/cfRNA sequencing read or a normal-plasma cfDNA/cfRNA sequencing read, based on the methylation cfDNA/cfRNA sequencing data (e.g., obtained using a methylation sequencing method, such as Bisulfite sequencing) and cancer methylation markers. Next, a profile of the tumor-derived sequencing read counts may be constructed. The constructed tumor-derived sequencing read profile may then be normalized. The CNV status (e.g., gain or loss) of each genomic region may be inferred, and a diagnosis or prognosis can be made based on the inferred CNV profile of a subject.

In an aspect, the present disclosure provides a method for detecting copy number variants (CNVs) from a plurality of cell-free nucleic acids of a subject, the method comprising: obtaining a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and using methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises: classifying a sequencing read of the methylation sequencing data as a tumor-derived sequencing read or a normal sequencing read; constructing a profile of tumor-derived sequencing read counts, wherein constructing the profile comprises quantifying the plurality of tumor-derived sequencing reads at each of a plurality of genomic regions; normalizing the constructed profile of tumor-derived sequencing read counts, to produce a normalized profile of tumor-derived sequencing read counts; and inferring a CNV status for each of the plurality of genomic regions based on the normalized profile of tumor-derived sequencing read counts.

In some embodiments, classifying a sequencing read of the methylation sequencing data as a tumor-derived sequencing read or a normal sequencing read comprises at least one of: (i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and (ii) calculating a posterior probability for the sequencing read, and comparing the posterior probability to a posterior probability threshold, wherein a posterior probability that exceeds the posterior probability threshold indicates a tumor-derived sequencing read.

In some embodiments, classifying the sequencing read as a tumor-derived sequencing read or a normal sequencing read further comprises: calculating a class-specific likelihood for the sequencing read.

In some embodiments, constructing the profile of tumor-derived sequencing read counts comprises excluding all of the plurality of sequencing reads classified as a normal sequencing read.

In some embodiments, constructing the profile of tumor-derived sequencing read counts comprises dividing at least a portion of the human genome into the plurality of genomic regions, the plurality of genomic regions comprising non-overlapping bins, according to a genome-wide segmentation strategy.

In some embodiments, the non-overlapping bins have a fixed size.

In some embodiments, the non-overlapping bins vary in size.

In some embodiments, normalizing the constructed profile of the tumor-derived sequencing read counts comprises calculating a fraction of tumor-derived cell-free nucleic acids in each of the plurality of genomic regions of the constructed profile.

In some embodiments, normalizing the constructed profile of the tumor-derived sequencing read counts comprises performing a bias correction of the constructed profile.

In some embodiments, performing the bias correction reduces bias attributable to at least one of: GC contents, sequencing read mapping, sequencing library construction, and sequencing platforms.

In some embodiments, performing the bias correction comprises comparing the constructed profile to a reference profile.

In some embodiments, the reference profile is a matched normal sample comprising genomic DNA from white blood cells obtained from a same blood sample as the plurality of cell-free nucleic acids.

In some embodiments, the reference profile is constructed from one or more cfDNA samples obtained from healthy subjects.

In some embodiments, the reference profile is constructed from certain genomic regions within a same sample.

In some embodiments, normalizing the constructed profile of tumor-derived sequencing read counts comprises measuring log ratios between case and control samples for each of the plurality of genomic regions.

In some embodiments, the method further comprises detecting a cancer of the subject based on the plurality of inferred CNV statuses.

In some embodiments, the cancer is detected based on a fraction of one or more genomic regions having tumor-derived sequencing read counts, and the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

In some embodiments, the method further comprises using the CNV status for treatment monitoring of the subject. In some embodiments, the method further comprises using the CNV status for patient stratification of the subject. In some embodiments, the method further comprises using CNV status for tracing a tissue-of-origin of the plurality of cell-free nucleic acids.

In some embodiments, the method further comprises identifying the at least one cancer methylation marker by processing methylation data of solid tumor samples, normal tissue samples, cell-free nucleic acid samples, or a combination thereof, obtained from one or more additional subjects.

In some embodiments, the at least one cancer methylation marker comprises epialleles, individual CpG sites, genomic regions, or a combination thereof.

In some embodiments, processing the methylation data comprises identifying the at least one cancer methylation marker based on a differential methylation of the at least one cancer methylation marker between the solid tumor samples, the normal tissue samples, the cell-free nucleic acid samples, or the combination thereof.

In some embodiments, the one or more additional subjects comprise one or more cancer patients and one or more normal subjects.

In some embodiments, processing the methylation data comprises identifying the at least one cancer methylation marker based on a differential methylation of the at least one cancer methylation marker between samples obtained from the one or more cancer patients and samples obtained from the one or more normal subjects.

In some embodiments, the plurality of cell-free nucleic acids comprises cell-free deoxyribonucleic acid (cfDNA). In some embodiments, the plurality of cell-free nucleic acids comprises cell-free ribonucleic acid (cfRNA).

In some embodiments, the method further comprises subjecting the plurality of cell-free nucleic acids to amplification. In some embodiments, the amplification comprises polymerase chain reaction (PCR). In some embodiments, the method further comprises processing the inferred plurality of CNV statuses against a reference. In some embodiments, the reference comprises a second plurality of CNV statuses detected from a plurality of cell-free nucleic acids of the same subject or one or more additional subjects. In some embodiments, the reference profile comprises CNV statuses in certain genomic regions within a same sample.

In some embodiments, the plurality of cell-free nucleic acids is obtained from a bodily sample of the subject. In some embodiments, the bodily sample is selected from the group consisting of plasma, serum, bone marrow, cerebral spinal fluid, pleural fluid, saliva, stool, and urine. In some embodiments, the method further comprises processing the inferred plurality of CNV statuses to generate a likelihood of the subject as having or being suspected of having a disease or disorder. In some embodiments, the disease or disorder is a cancer. In some embodiments, the cancer is selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, and prostate cancer. In some embodiments, the subject is asymptomatic of the disease or disorder.

In some embodiments, the method further comprises generating a likelihood of the subject as having or being suspected of having a disease or disorder with a sensitivity of at least about 60%. In some embodiments, the method further comprises generating a likelihood of the subject as having or being suspected of having a disease or disorder with a sensitivity of at least about 70%. In some embodiments, the method further comprises generating a likelihood of the subject as having or being suspected of having a disease or disorder with a sensitivity of at least about 80%. In some embodiments, the method further comprises generating a likelihood of the subject as having or being suspected of having a disease or disorder with a sensitivity of at least about 90%. In some embodiments, the method further comprises generating a likelihood of the subject as having or being suspected of having a disease or disorder with a sensitivity of at least about 95%.

In some embodiments, the method further comprises generating a likelihood of the subject as having or being suspected of having a disease or disorder with a specificity of at least about 60%. In some embodiments, the method further comprises generating a likelihood of the subject as having or being suspected of having a disease or disorder with a specificity of at least about 70%. In some embodiments, the method further comprises generating a likelihood of the subject as having or being suspected of having a disease or disorder with a specificity of at least about 80%. In some embodiments, the method further comprises generating a likelihood of the subject as having or being suspected of having a disease or disorder with a specificity of at least about 90%. In some embodiments, the method further comprises generating a likelihood of the subject as having or being suspected of having a disease or disorder with a specificity of at least about 95%.

In some embodiments, the method further comprises generating a likelihood of the subject as having or being suspected of having a disease or disorder with an accuracy of at least about 60%. In some embodiments, the method further comprises generating a likelihood of the subject as having or being suspected of having a disease or disorder with an accuracy of at least about 70%. In some embodiments, the method further comprises generating a likelihood of the subject as having or being suspected of having a disease or disorder with an accuracy of at least about 80%. In some embodiments, the method further comprises generating a likelihood of the subject as having or being suspected of having a disease or disorder with an accuracy of at least about 90%. In some embodiments, the method further comprises generating a likelihood of the subject as having or being suspected of having a disease or disorder with an accuracy of at least about 95%.

In some embodiments, the method further comprises generating a likelihood of the subject as having or being suspected of having a disease or disorder with a positive predictive value of at least about 60%. In some embodiments, the method further comprises generating a likelihood of the subject as having or being suspected of having a disease or disorder with a positive predictive value of at least about 70%. In some embodiments, the method further comprises generating a likelihood of the subject as having or being suspected of having a disease or disorder with a positive predictive value of at least about 80%. In some embodiments, the method further comprises generating a likelihood of the subject as having or being suspected of having a disease or disorder with a positive predictive value of at least about 90%. In some embodiments, the method further comprises generating a likelihood of the subject as having or being suspected of having a disease or disorder with a positive predictive value of at least about 95%.

In some embodiments, the method further comprises generating a likelihood of the subject as having or being suspected of having a disease or disorder with a negative predictive value of at least about 60%. In some embodiments, the method further comprises generating a likelihood of the subject as having or being suspected of having a disease or disorder with a negative predictive value of at least about 70%. In some embodiments, the method further comprises generating a likelihood of the subject as having or being suspected of having a disease or disorder with a negative predictive value of at least about 80%. In some embodiments, the method further comprises generating a likelihood of the subject as having or being suspected of having a disease or disorder with a negative predictive value of at least about 90%. In some embodiments, the method further comprises generating a likelihood of the subject as having or being suspected of having a disease or disorder with a negative predictive value of at least about 95%.

In some embodiments, the method further comprises generating a likelihood of the subject as having or being suspected of having a disease or disorder with an area under the receiver-operating characteristic (AUROC) of at least about 0.60. In some embodiments, the method further comprises generating a likelihood of the subject as having or being suspected of having a disease or disorder with an AUROC of at least about 0.70. In some embodiments, the method further comprises generating a likelihood of the subject as having or being suspected of having a disease or disorder with an AUROC of at least about 0.80. In some embodiments, the method further comprises generating a likelihood of the subject as having or being suspected of having a disease or disorder with an AUROC of at least about 0.90. In some embodiments, the method further comprises generating a likelihood of the subject as having or being suspected of having a disease or disorder with an AUROC of at least about 0.95.

In some embodiments, the method further comprises sequencing the plurality of cell-free nucleic acids or derivatives thereof to yield the plurality of sequencing reads. In some embodiments, the inferred plurality of CNV statuses comprises cancer somatic driver mutations.

In another aspect, the present disclosure provides a system for detecting copy number variants (CNVs) from a plurality of cell-free nucleic acids of a subject, the system comprising: a memory; one or more processors communicatively coupled to the memory, the one or more processors individually or collectively programmed to: obtain a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and use methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises: classifying a sequencing read of the methylation sequencing data as a tumor-derived sequencing read or a normal sequencing read; constructing a profile of tumor-derived sequencing read counts, wherein constructing the profile comprises quantifying the plurality of tumor-derived sequencing reads at each of a plurality of genomic regions; normalizing the constructed profile of tumor-derived sequencing read counts, to produce a normalized profile of tumor-derived sequencing read counts; and inferring a CNV status for each of the plurality of genomic regions based on the normalized profile of tumor-derived sequencing read counts.

In some embodiments, classifying a sequencing read of the methylation sequencing data as a tumor-derived sequencing read or a normal sequencing read comprises at least one of: (i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and (ii) calculating a posterior probability for the sequencing read, and comparing the posterior probability to a posterior probability threshold, wherein a posterior probability that exceeds the posterior probability threshold indicates a tumor-derived sequencing read.

In some embodiments, classifying the sequencing read as a tumor-derived sequencing read or a normal sequencing read further comprises: calculating a class-specific likelihood for the sequencing read.

In some embodiments, constructing the profile of tumor-derived sequencing read counts comprises excluding all of the plurality of sequencing reads classified as a normal sequencing read.

In some embodiments, constructing the profile of tumor-derived sequencing read counts comprises dividing at least a portion of the human genome into the plurality of genomic regions, the plurality of genomic regions comprising non-overlapping bins, according to a genome-wide segmentation strategy.

In some embodiments, the non-overlapping bins have a fixed size.

In some embodiments, the non-overlapping bins vary in size.

In some embodiments, normalizing the constructed profile of the tumor-derived sequencing read counts comprises calculating a fraction of tumor-derived cell-free nucleic acids in each of the plurality of genomic regions of the constructed profile.

In some embodiments, normalizing the constructed profile of the tumor-derived sequencing read counts comprises performing a bias correction of the constructed profile.

In some embodiments, performing the bias correction reduces bias attributable to at least one of: GC contents, sequencing read mapping, sequencing library construction, and sequencing platforms.

In some embodiments, performing the bias correction comprises comparing the constructed profile to a reference profile.

In some embodiments, the reference profile is a matched normal sample comprising genomic DNA from white blood cells obtained from a same blood sample as the plurality of cell-free nucleic acids.

In some embodiments, the reference profile is constructed from one or more cfDNA samples obtained from healthy subjects.

In some embodiments, the reference profile is constructed from certain genomic regions within a same sample.

In some embodiments, normalizing the constructed profile of tumor-derived sequencing read counts comprises measuring log ratios between case and control samples for each of the plurality of genomic regions.

In some embodiments, the one or more processors are programmed to detect a cancer of the subject based on the plurality of inferred CNV statuses.

In some embodiments, the one or more processors are individually or collectively programmed to further use the CNV status for treatment monitoring of the subject.

In some embodiments, the one or more processors are individually or collectively programmed to further use the CNV status for patient stratification of the subject.

In some embodiments, the one or more processors are individually or collectively programmed to further use the CNV status for tracing a tissue-of-origin of the plurality of cell-free nucleic acids.

In some embodiments, the one or more processors are individually or collectively programmed to further identify the at least one cancer methylation marker by processing methylation data of solid tumor samples, normal tissue samples, cell-free nucleic acid samples, or a combination thereof, obtained from one or more additional subjects.

In some embodiments, the at least one cancer methylation marker comprises epialleles, individual CpG sites, genomic regions, or a combination thereof.

In some embodiments, processing the methylation data comprises identifying the at least one cancer methylation marker based on a differential methylation of the at least one cancer methylation marker between the solid tumor samples, the normal tissue samples, the cell-free nucleic acid samples, or the combination thereof.

In some embodiments, the one or more additional subjects comprise one or more cancer patients and one or more normal subjects.

In some embodiments, processing the methylation data comprises identifying the at least one cancer methylation marker based on a differential methylation of the at least one cancer methylation marker between samples obtained from the one or more cancer patients and samples obtained from the one or more normal subjects.

In some embodiments, the cancer is detected based on a fraction of one or more genomic regions having tumor-derived sequencing read counts, and wherein the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

In some embodiments, the plurality of cell-free nucleic acids comprises cell-free deoxyribonucleic acid (cfDNA). In some embodiments, the plurality of cell-free nucleic acids comprises cell-free ribonucleic acid (cfRNA).

In some embodiments, the one or more processors are programmed to direct the plurality of cell-free nucleic acids to be subjected to amplification. In some embodiments, the amplification comprises polymerase chain reaction (PCR). In some embodiments, the one or more processors are programmed to process the inferred plurality of CNV statuses against a reference. In some embodiments, the reference comprises a second plurality of CNV statuses detected from a plurality of cell-free nucleic acids of the same subject or one or more additional subjects.

In some embodiments, the plurality of cell-free nucleic acids is obtained from a bodily sample of the subject. In some embodiments, the bodily sample is selected from the group consisting of plasma, serum, bone marrow, cerebral spinal fluid, pleural fluid, saliva, stool, and urine. In some embodiments, the one or more processors are programmed to process the inferred plurality of CNV statuses to generate a likelihood of the subject as having or being suspected of having a disease or disorder. In some embodiments, the disease or disorder is a cancer. In some embodiments, the cancer is selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, and prostate cancer. In some embodiments, the subject is asymptomatic of the disease or disorder.

In some embodiments, the one or more processors are programmed to generate a likelihood of the subject as having or being suspected of having a disease or disorder with a sensitivity of at least about 60%. In some embodiments, the one or more processors are programmed to generate a likelihood of the subject as having or being suspected of having a disease or disorder with a sensitivity of at least about 70%. In some embodiments, the one or more processors are programmed to generate a likelihood of the subject as having or being suspected of having a disease or disorder with a sensitivity of at least about 80%. In some embodiments, the one or more processors are programmed to generate a likelihood of the subject as having or being suspected of having a disease or disorder with a sensitivity of at least about 90%. In some embodiments, the one or more processors are programmed to generate a likelihood of the subject as having or being suspected of having a disease or disorder with a sensitivity of at least about 95%.

In some embodiments, the one or more processors are programmed to generate a likelihood of the subject as having or being suspected of having a disease or disorder with a specificity of at least about 60%. In some embodiments, the one or more processors are programmed to generate a likelihood of the subject as having or being suspected of having a disease or disorder with a specificity of at least about 70%. In some embodiments, the one or more processors are programmed to generate a likelihood of the subject as having or being suspected of having a disease or disorder with a specificity of at least about 80%. In some embodiments, the one or more processors are programmed to generate a likelihood of the subject as having or being suspected of having a disease or disorder with a specificity of at least about 90%. In some embodiments, the one or more processors are programmed to generate a likelihood of the subject as having or being suspected of having a disease or disorder with a specificity of at least about 95%.

In some embodiments, the one or more processors are programmed to generate a likelihood of the subject as having or being suspected of having a disease or disorder with an accuracy of at least about 60%. In some embodiments, the one or more processors are programmed to generate a likelihood of the subject as having or being suspected of having a disease or disorder with an accuracy of at least about 70%. In some embodiments, the one or more processors are programmed to generate a likelihood of the subject as having or being suspected of having a disease or disorder with an accuracy of at least about 80%. In some embodiments, the one or more processors are programmed to generate a likelihood of the subject as having or being suspected of having a disease or disorder with an accuracy of at least about 90%. In some embodiments, the one or more processors are programmed to generate a likelihood of the subject as having or being suspected of having a disease or disorder with an accuracy of at least about 95%.

In some embodiments, the one or more processors are programmed to generate a likelihood of the subject as having or being suspected of having a disease or disorder with a positive predictive value of at least about 60%. In some embodiments, the one or more processors are programmed to generate a likelihood of the subject as having or being suspected of having a disease or disorder with a positive predictive value of at least about 70%. In some embodiments, the one or more processors are programmed to generate a likelihood of the subject as having or being suspected of having a disease or disorder with a positive predictive value of at least about 80%. In some embodiments, the one or more processors are programmed to generate a likelihood of the subject as having or being suspected of having a disease or disorder with a positive predictive value of at least about 90%. In some embodiments, the one or more processors are programmed to generate a likelihood of the subject as having or being suspected of having a disease or disorder with a positive predictive value of at least about 95%.

In some embodiments, the one or more processors are programmed to generate a likelihood of the subject as having or being suspected of having a disease or disorder with a negative predictive value of at least about 60%. In some embodiments, the one or more processors are programmed to generate a likelihood of the subject as having or being suspected of having a disease or disorder with a negative predictive value of at least about 70%. In some embodiments, the one or more processors are programmed to generate a likelihood of the subject as having or being suspected of having a disease or disorder with a negative predictive value of at least about 80%. In some embodiments, the one or more processors are programmed to generate a likelihood of the subject as having or being suspected of having a disease or disorder with a negative predictive value of at least about 90%. In some embodiments, the one or more processors are programmed to generate a likelihood of the subject as having or being suspected of having a disease or disorder with a negative predictive value of at least about 95%.

In some embodiments, the one or more processors are programmed to generate a likelihood of the subject as having or being suspected of having a disease or disorder with an area under the receiver-operating characteristic (AUROC) of at least about 0.60. In some embodiments, the one or more processors are programmed to generate a likelihood of the subject as having or being suspected of having a disease or disorder with an AUROC of at least about 0.70. In some embodiments, the one or more processors are programmed to generate a likelihood of the subject as having or being suspected of having a disease or disorder with an AUROC of at least about 0.80. In some embodiments, the one or more processors are programmed to generate a likelihood of the subject as having or being suspected of having a disease or disorder with an AUROC of at least about 0.90. In some embodiments, the one or more processors are programmed to generate a likelihood of the subject as having or being suspected of having a disease or disorder with an AUROC of at least about 0.95.

In some embodiments, the one or more processors are programmed to sequence the plurality of cell-free nucleic acids or derivatives thereof to yield the plurality of sequencing reads. In some embodiments, the inferred plurality of CNV statuses comprises cancer somatic driver mutations.

In another aspect, the present disclosure provides a non-transitory computer-readable storage medium storing a set of instructions that, when executed, cause one or more processors to detect copy number variants (CNVs) from a plurality of cell-free nucleic acids of a subject, the set of instructions comprising instructions to: obtain a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and use methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises: classifying a sequencing read of the methylation sequencing data as a tumor-derived sequencing read or a normal sequencing read; constructing a profile of tumor-derived sequencing read counts, wherein constructing the profile comprises quantifying the plurality of tumor-derived sequencing reads at each of a plurality of genomic regions; normalizing the constructed profile of tumor-derived sequencing read counts, to produce a normalized profile of tumor-derived sequencing read counts; and inferring a CNV status for each of the plurality of genomic regions based on the normalized profile of tumor-derived sequencing read counts.

In some embodiments, classifying a sequencing read of the methylation sequencing data as a tumor-derived sequencing read or a normal sequencing read comprises at least one of: (i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and (ii) calculating a posterior probability for the sequencing read, and comparing the posterior probability to a posterior probability threshold, wherein a posterior probability that exceeds the posterior probability threshold indicates a tumor-derived sequencing read.

In some embodiments, classifying the sequencing read as a tumor-derived sequencing read or a normal sequencing read further comprises: calculating a class-specific likelihood for the sequencing read.

In some embodiments, constructing the profile of tumor-derived sequencing read counts comprises excluding all of the plurality of sequencing reads classified as a normal sequencing read.

In some embodiments, constructing the profile of tumor-derived sequencing read counts comprises dividing at least a portion of the human genome into the plurality of genomic regions, the plurality of genomic regions comprising non-overlapping bins, according to a genome-wide segmentation strategy.

In some embodiments, the non-overlapping bins have a fixed size.

In some embodiments, the non-overlapping bins vary in size.

In some embodiments, normalizing the constructed profile of the tumor-derived sequencing read counts comprises calculating a fraction of tumor-derived cell-free nucleic acids in each of the plurality of genomic regions of the constructed profile.

In some embodiments, normalizing the constructed profile of the tumor-derived sequencing read counts comprises performing a bias correction of the constructed profile.

In some embodiments, performing the bias correction reduces bias attributable to at least one of: GC contents, sequencing read mapping, sequencing library construction, and sequencing platforms.

In some embodiments, performing the bias correction comprises comparing the constructed profile to a reference profile.

In some embodiments, the reference profile is a matched normal sample comprising genomic DNA from white blood cells obtained from a same blood sample as the plurality of cell-free nucleic acids.

In some embodiments, the reference profile is constructed from one or more cfDNA samples obtained from healthy subjects.

In some embodiments, the reference profile is constructed from certain genomic regions within a same sample.

In some embodiments, normalizing the constructed profile of tumor-derived sequencing read counts comprises measuring log ratios between case and control samples for each of the plurality of genomic regions.

In some embodiments, the set of instructions comprises instructions to detect a cancer of the subject based on the plurality of inferred CNV statuses.

In some embodiments, the cancer is detected based on a fraction of one or more genomic regions having tumor-derived sequencing read counts, and wherein the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

In some embodiments, the set of instructions comprises instructions to use the CNV status for treatment monitoring of the subject.

In some embodiments, the set of instructions comprises instructions to use the CNV status for patient stratification of the subject.

In some embodiments, the set of instructions comprises instructions to use the CNV status for tracing a tissue-of-origin of the plurality of cell-free nucleic acids.

In some embodiments, the set of instructions comprises instructions to identify the at least one cancer methylation marker by processing methylation data of solid tumor samples, normal tissue samples, cell-free nucleic acid samples, or a combination thereof, obtained from one or more additional subjects.

In some embodiments, the at least one cancer methylation marker comprises epialleles, individual CpG sites, genomic regions, or a combination thereof.

In some embodiments, processing the methylation data comprises identifying the at least one cancer methylation marker based on a differential methylation of the at least one cancer methylation marker between the solid tumor samples, the normal tissue samples, the cell-free nucleic acid samples, or the combination thereof.

In some embodiments, the one or more additional subjects comprise one or more cancer patients and one or more normal subjects.

In some embodiments, processing the methylation data comprises identifying the at least one cancer methylation marker based on a differential methylation of the at least one cancer methylation marker between samples obtained from the one or more cancer patients and samples obtained from the one or more normal subjects.

In some embodiments, the plurality of cell-free nucleic acids comprises cell-free deoxyribonucleic acid (cfDNA). In some embodiments, the plurality of cell-free nucleic acids comprises cell-free ribonucleic acid (cfRNA).

In some embodiments, the set of instructions comprises instructions to direct the plurality of cell-free nucleic acids to be subjected to amplification. In some embodiments, the amplification comprises polymerase chain reaction (PCR). In some embodiments, the set of instructions comprises instructions to process the inferred plurality of CNV statuses against a reference. In some embodiments, the reference comprises a second plurality of CNV statuses detected from a plurality of cell-free nucleic acids of the same subject or one or more additional subjects.

In some embodiments, the plurality of cell-free nucleic acids is obtained from a bodily sample of the subject. In some embodiments, the bodily sample is selected from the group consisting of plasma, serum, bone marrow, cerebral spinal fluid, pleural fluid, saliva, stool, and urine. In some embodiments, the set of instructions comprises instructions to process the inferred plurality of CNV statuses to generate a likelihood of the subject as having or being suspected of having a disease or disorder. In some embodiments, the disease or disorder is a cancer. In some embodiments, the cancer is selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, and prostate cancer. In some embodiments, the subject is asymptomatic of the disease or disorder.

In some embodiments, the set of instructions comprises instructions to generate a likelihood of the subject as having or being suspected of having a disease or disorder with a sensitivity of at least about 60%. In some embodiments, the set of instructions comprises instructions to generate a likelihood of the subject as having or being suspected of having a disease or disorder with a sensitivity of at least about 70%. In some embodiments, the set of instructions comprises instructions to generate a likelihood of the subject as having or being suspected of having a disease or disorder with a sensitivity of at least about 80%. In some embodiments, the set of instructions comprises instructions to generate a likelihood of the subject as having or being suspected of having a disease or disorder with a sensitivity of at least about 90%. In some embodiments, the set of instructions comprises instructions to generate a likelihood of the subject as having or being suspected of having a disease or disorder with a sensitivity of at least about 95%.

In some embodiments, the set of instructions comprises instructions to generate a likelihood of the subject as having or being suspected of having a disease or disorder with a specificity of at least about 60%. In some embodiments, the set of instructions comprises instructions to generate a likelihood of the subject as having or being suspected of having a disease or disorder with a specificity of at least about 70%. In some embodiments, the set of instructions comprises instructions to generate a likelihood of the subject as having or being suspected of having a disease or disorder with a specificity of at least about 80%. In some embodiments, the set of instructions comprises instructions to generate a likelihood of the subject as having or being suspected of having a disease or disorder with a specificity of at least about 90%. In some embodiments, the set of instructions comprises instructions to generate a likelihood of the subject as having or being suspected of having a disease or disorder with a specificity of at least about 95%.

In some embodiments, the set of instructions comprises instructions to generate a likelihood of the subject as having or being suspected of having a disease or disorder with an accuracy of at least about 60%. In some embodiments, the set of instructions comprises instructions to generate a likelihood of the subject as having or being suspected of having a disease or disorder with an accuracy of at least about 70%. In some embodiments, the set of instructions comprises instructions to generate a likelihood of the subject as having or being suspected of having a disease or disorder with an accuracy of at least about 80%. In some embodiments, the set of instructions comprises instructions to generate a likelihood of the subject as having or being suspected of having a disease or disorder with an accuracy of at least about 90%. In some embodiments, the set of instructions comprises instructions to generate a likelihood of the subject as having or being suspected of having a disease or disorder with an accuracy of at least about 95%.

In some embodiments, the set of instructions comprises instructions to generate a likelihood of the subject as having or being suspected of having a disease or disorder with a positive predictive value of at least about 60%. In some embodiments, the set of instructions comprises instructions to generate a likelihood of the subject as having or being suspected of having a disease or disorder with a positive predictive value of at least about 70%. In some embodiments, the set of instructions comprises instructions to generate a likelihood of the subject as having or being suspected of having a disease or disorder with a positive predictive value of at least about 80%. In some embodiments, the set of instructions comprises instructions to generate a likelihood of the subject as having or being suspected of having a disease or disorder with a positive predictive value of at least about 90%. In some embodiments, the set of instructions comprises instructions to generate a likelihood of the subject as having or being suspected of having a disease or disorder with a positive predictive value of at least about 95%.

In some embodiments, the set of instructions comprises instructions to generate a likelihood of the subject as having or being suspected of having a disease or disorder with a negative predictive value of at least about 60%. In some embodiments, the set of instructions comprises instructions to generate a likelihood of the subject as having or being suspected of having a disease or disorder with a negative predictive value of at least about 70%. In some embodiments, the set of instructions comprises instructions to generate a likelihood of the subject as having or being suspected of having a disease or disorder with a negative predictive value of at least about 80%. In some embodiments, the set of instructions comprises instructions to generate a likelihood of the subject as having or being suspected of having a disease or disorder with a negative predictive value of at least about 90%. In some embodiments, the set of instructions comprises instructions to generate a likelihood of the subject as having or being suspected of having a disease or disorder with a negative predictive value of at least about 95%.

In some embodiments, the set of instructions comprises instructions to generate a likelihood of the subject as having or being suspected of having a disease or disorder with an area under the receiver-operating characteristic (AUROC) of at least about 0.60. In some embodiments, the set of instructions comprises instructions to generate a likelihood of the subject as having or being suspected of having a disease or disorder with an AUROC of at least about 0.70. In some embodiments, the set of instructions comprises instructions to generate a likelihood of the subject as having or being suspected of having a disease or disorder with an AUROC of at least about 0.80. In some embodiments, the set of instructions comprises instructions to generate a likelihood of the subject as having or being suspected of having a disease or disorder with an AUROC of at least about 0.90. In some embodiments, the set of instructions comprises instructions to generate a likelihood of the subject as having or being suspected of having a disease or disorder with an AUROC of at least about 0.95.

In some embodiments, the set of instructions comprises instructions to sequence the plurality of cell-free nucleic acids or derivatives thereof to yield the plurality of sequencing reads. In some embodiments, the inferred plurality of CNV statuses comprises cancer somatic driver mutations.

In another aspect, the present disclosure provides a method for detecting fetal copy number variants (CNVs) from a plurality of cell-free nucleic acids of a maternal sample of a pregnant subject, the method comprising: obtaining a plurality of sequencing reads derived by sequencing the plurality of cell-free nucleic acids, wherein the plurality of sequencing reads comprises (i) a plurality of fetal-derived sequencing reads corresponding to fetal-derived cell-free nucleic acids of the plurality of cell-free nucleic acids and (ii) a plurality of normal sequencing reads corresponding to normal cell-free nucleic acids of the plurality of cell-free nucleic acids; and using methylation sequencing data of the plurality of cell-free nucleic acids and at least one fetal methylation marker to distinguish the plurality of fetal-derived sequencing reads from the plurality of normal sequencing reads, wherein distinguishing the plurality of fetal-derived sequencing reads from the plurality of normal sequencing reads comprises: classifying a sequencing read of the methylation sequencing data as a fetal-derived sequencing read or a normal sequencing read; constructing a profile of fetal-derived sequencing read counts, wherein constructing the profile comprises quantifying the plurality of fetal-derived sequencing reads at each of a plurality of genomic regions; normalizing the constructed profile of fetal-derived sequencing read counts, to produce a normalized profile of fetal-derived sequencing read counts; and inferring a CNV status for each of the plurality of genomic regions based on the normalized profile of fetal-derived sequencing read counts.

In some embodiments, classifying a sequencing read of the methylation sequencing data as a fetal-derived sequencing read or a normal sequencing read comprises at least one of: (i) calculating a likelihood ratio for the sequencing read, and comparing the likelihood ratio to a likelihood ratio threshold, wherein a likelihood ratio that exceeds the likelihood ratio threshold indicates a fetal-derived sequencing read; and (ii) calculating a posterior probability for the sequencing read, and comparing the posterior probability to a posterior probability threshold, wherein a posterior probability that exceeds the posterior probability threshold indicates a fetal-derived sequencing read.

In some embodiments, classifying the sequencing read as a fetal-derived sequencing read or a normal sequencing read further comprises: calculating a class-specific likelihood for the sequencing read.

In some embodiments, constructing the profile of fetal-derived sequencing read counts comprises excluding all of the plurality of sequencing reads classified as a normal sequencing read.

In some embodiments, constructing the profile of fetal-derived sequencing read counts comprises dividing at least a portion of the human genome into the plurality of genomic regions, the plurality of genomic regions comprising non-overlapping bins, according to a genome-wide segmentation strategy.

In some embodiments, the non-overlapping bins have a fixed size.

In some embodiments, the non-overlapping bins vary in size.

In some embodiments, normalizing the constructed profile of the fetal-derived sequencing read counts comprises calculating a fraction of fetal-derived cell-free nucleic acids in each of the plurality of genomic regions of the constructed profile.

In some embodiments, normalizing the constructed profile of the fetal-derived sequencing read counts comprises performing a bias correction of the constructed profile.

In some embodiments, performing the bias correction reduces bias attributable to at least one of: GC contents, sequencing read mapping, sequencing library construction, and sequencing platforms.

In some embodiments, performing the bias correction comprises comparing the constructed profile to a reference profile.

In some embodiments, the reference profile is constructed from one or more cfDNA samples obtained from pregnant subjects with a healthy fetus.

In some embodiments, normalizing the constructed profile of fetal-derived sequencing read counts comprises measuring log ratios between case and control samples for each of the plurality of genomic regions.

In some embodiments, the method further comprises detecting a fetal anomaly of a fetus of the pregnant subject based on the plurality of inferred CNV statuses.

In some embodiments, the fetal anomaly of the fetus is detected based on a fraction of one or more genomic regions having fetal-derived sequencing read counts, and the detecting comprises using a fraction of the plurality of genomic regions having abnormal sequencing read counts as a fetal anomaly indicator score, wherein a genomic region is determined to have an abnormal sequencing read count based on a log ratio of the inferred CNV status of the genomic region.

In some embodiments, the plurality of cell-free nucleic acids comprises cell-free deoxyribonucleic acid (cfDNA). In some embodiments, the plurality of cell-free nucleic acids comprises cell-free ribonucleic acid (cfRNA).

In some embodiments, the method further comprises subjecting the plurality of cell-free nucleic acids to amplification. In some embodiments, the amplification comprises polymerase chain reaction (PCR). In some embodiments, the method further comprises processing the inferred plurality of CNV statuses against a reference. In some embodiments, the reference comprises a second plurality of CNV statuses detected from a plurality of cell-free nucleic acids of one or more additional pregnant subjects.

In some embodiments, the plurality of cell-free nucleic acids is obtained from a bodily sample of the pregnant subject. In some embodiments, the bodily sample is selected from the group consisting of plasma, serum, bone marrow, cerebral spinal fluid, pleural fluid, saliva, stool, and urine. In some embodiments, the method further comprises processing the inferred plurality of CNV statuses to generate a likelihood of the pregnant subject or a fetus of the pregnant subject as having or being suspected of having a disease or disorder. In some embodiments, the disease or disorder comprises a fetal anomaly (e.g., a fetal aneuploidy). In some embodiments, the fetal aneuploidy is Down Syndrome. In some embodiments, the method further comprises sequencing the plurality of cell-free nucleic acids or derivatives thereof to yield the plurality of sequencing reads.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. It is specifically contemplated that any limitation discussed with respect to one embodiment of the invention may apply to any other embodiment of the invention. Furthermore, any system or storage medium or other component of the invention may be used in any method of the invention, and any method of the invention may be used to produce or to utilize any component of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### DESCRIPTION OF FIGURES

For a more complete understanding of the present invention, reference is now made to the following descriptions taken in conjunction with the accompanying figures, in which:

FIG. 1 illustrates examples of aspects of a comparison between cell-free copy number variation (cfCNV) inference methods, according to a disclosed embodiment.

FIG. 2 illustrates examples of aspects of a method for detecting CNVs in one or more cfDNA samples, according to a disclosed embodiment.

FIG. 3 illustrates examples of concepts associated with distinguishing tumor-derived sequencing reads from normal sequencing reads in cfDNA, according to a disclosed embodiment.

FIG. 4 illustrates an example of cancer markers identified by a method for discovery of markers that cover the genome, according to a disclosed embodiment, including a distribution of numbers of discovered markers within bins of 1M bp throughout the entire genome.

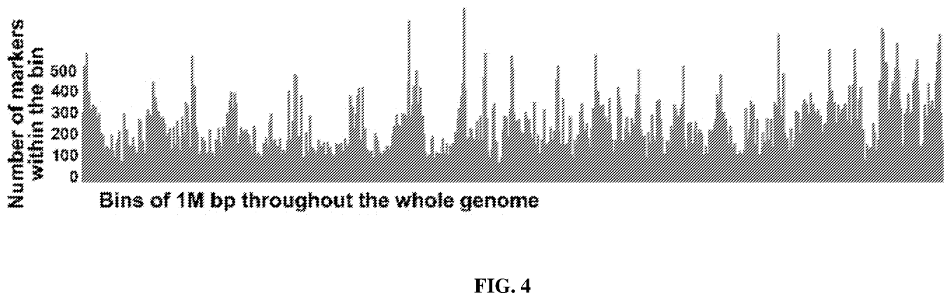

FIG. 5 illustrates different methylation patterns of a marker for a tumor type T, which are defined at different resolutions at levels of (A) epialleles, (B) CpG sites, and (C) a genomic region, according to a disclosed embodiment. These methylation patterns can be defined for a normal class similarly.

FIG. 6 illustrates an example of a method for calculating the class-specific likelihoods of a given cfDNA sequencing read, according to a disclosed embodiment.

FIG. 7 illustrates an example of calculating a sequencing read's class-specific likelihoods, according to a disclosed embodiment.

FIG. 8 illustrates an example in which the False Positive Rate (FPR) from the cfDNA of a healthy individual is extremely low for the vast majority of markers, according to a disclosed embodiment. FIG. 8 shows (A) an FPR histogram of each cancer-specific marker estimated from a healthy individual's cfDNA sample and (B) a zoomed-out view of the histogram of (A) that excludes the bar with FPR=0.

FIG. 9A illustrates examples of aspects of results achieved by a disclosed embodiment.

FIG. 9B illustrates examples of aspects of results achieved by a disclosed embodiment. The CNV profile obtained from cfDNA samples of pregnant subjects by a cfCNV method disclosed herein can detect the same duplication regions (e.g., indicative of CNV gain) and deletion regions (e.g., indicative of CNV loss) as those found in a solid placenta tissue sample from the same subject. In comparison, a traditional CNV method (e.g., total read count-based method) fails to do so.

FIG. 10 illustrates examples of components of a system for performing methods of the present disclosure, according to a disclosed embodiment.

FIG. 11 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a healthy subject, a patient with a disease or disorder (e.g., a cancer), a patient suspected of having a disease or disorder (e.g., a cancer), a pregnant female subject, or a female subject suspected of being pregnant. The subject may be displaying a symptom(s) indicative of a health or physiological state or condition of the subject, such as a cancer-related health or physiological state or condition of the subject. As an alternative, the subject can be asymptomatic with respect to such health or physiological state or condition.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

The present disclosure provides methods and systems for detecting or inferring quantitative measures of copy number variations, copy number alterations, or copy number polymorphisms (collectively referred to as Copy Number Variants (CNVs)) in cell-free nucleic acid samples, such as cell-free DNA (cfDNA) and/or cell-free RNA (cfRNA) samples, even in cases where an amount or level of CNVs in a cfDNA/cfRNA sample is low. Since cfDNA is often used for detecting CNVs, the present disclosure generally makes reference to cfDNA (without expressly making reference to cfRNA). However, it should be understood that the methods and systems provided herein may also be applied to other types of nucleic acids, such as cfRNA. Therefore, any references to “cfDNA” in the present disclosure may also expressly apply to other types of circulating nucleic acids.

In some embodiments, methods and systems of the present disclosure can be utilized to detect CNVs in an individual patient. In some embodiments, methods and systems of the present disclosure can be utilized to detect fetal CNVs from maternal blood.

In an aspect, the present disclosure provides a method for sensitively detecting CNVs in cfDNA samples, which may comprise using cfDNA methylation sequencing data and cancer methylation markers to distinguish tumor-derived sequencing reads from normal sequencing reads. Each cfDNA sequencing read among a plurality of cfDNA sequencing reads (e.g., containing cancer methylation markers) of a cfDNA sample may be classified as either corresponding to a tumor-derived cfDNA or a normal-plasma cfDNA, based on the methylation cfDNA sequencing data (e.g., obtained using a methylation sequencing method, such as Bisulfite sequencing) and cancer methylation markers. Based on the classification, only the set of tumor-derived sequencing reads of a cfDNA sample may be utilized to infer CNV. Next, a profile of the tumor-derived sequencing read counts may be constructed (e.g., by quantifying the tumor-derived sequencing read counts in each a plurality of genomic regions or bins). The constructed tumor-derived sequencing read profile may then be normalized. The CNV status (e.g., gain or loss) of each genomic region may be inferred, and a diagnosis or prognosis may be made based on the inferred CNV profile of a subject.

Detecting or inferring CNVs in cfDNA samples according to methods and systems of the present disclosure may be referred to herein as cell-free CNV (cfCNV) methods. The cfCNV methods and systems described herein may be capable of detecting CNVs with much higher sensitivity, specificity, and accuracy as compared to conventional sequencing read-count based CNV detection methods.

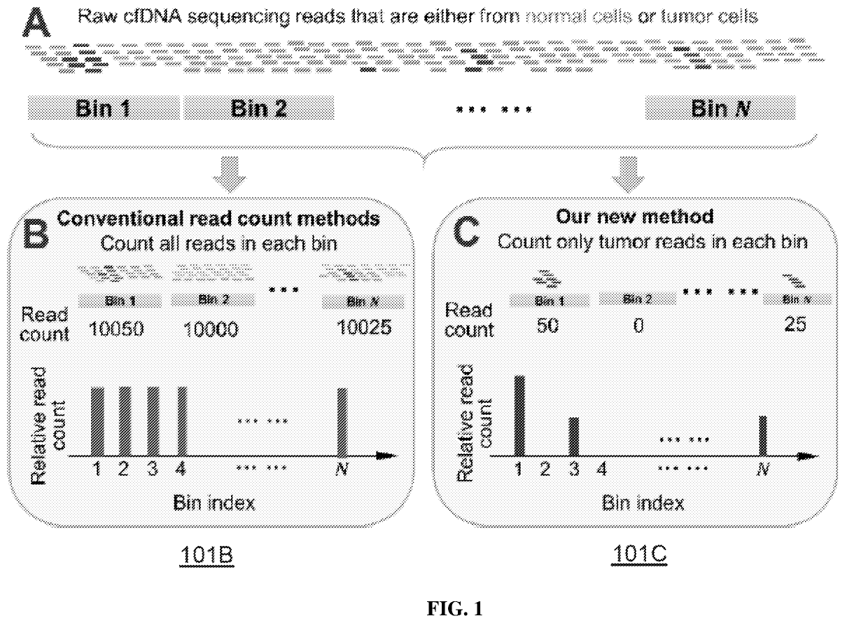

As an initial matter, the embodiments described herein, and the benefits provided by same, can be further understood by an examination of shortcomings of conventional methods. As mentioned, conventional RC methods may suffer a decrease in utility if the tumor-derived cfDNA fraction is low, because the signal from tumor-derived CNV is overwhelmed by the vast majority of normal (e.g., non-tumor) sequencing reads. This challenge is illustrated in FIG. 1, where tumor-derived sequencing reads (red) occupy a tiny fraction of all sequencing reads (e.g., a mixture comprising tumor-derived and normal sequencing reads). At panel 101A, FIG. 1 shows cfDNA reads that can comprise tumor-derived sequencing reads or normal sequencing reads. At panel 101B, FIG. 1 shows a conventional copy number inference approach, which counts all sequencing reads in each of a plurality of genomic regions (bins). For example, suppose that in the first bin, tumor cells duplicate a chromosome fragment, such that 50 tumor-derived sequencing reads are observed instead of 25 tumor-derived sequencing reads. However, there is a total of 10,050 reads observed in the first bin, so such a relatively small change may be typically regarded as noise. Hence, conventional RC methods may fail to accurately detect and call the CNV in such cases. Panel 101C of FIG. 1 illustrates concepts associated with embodiments described herein.

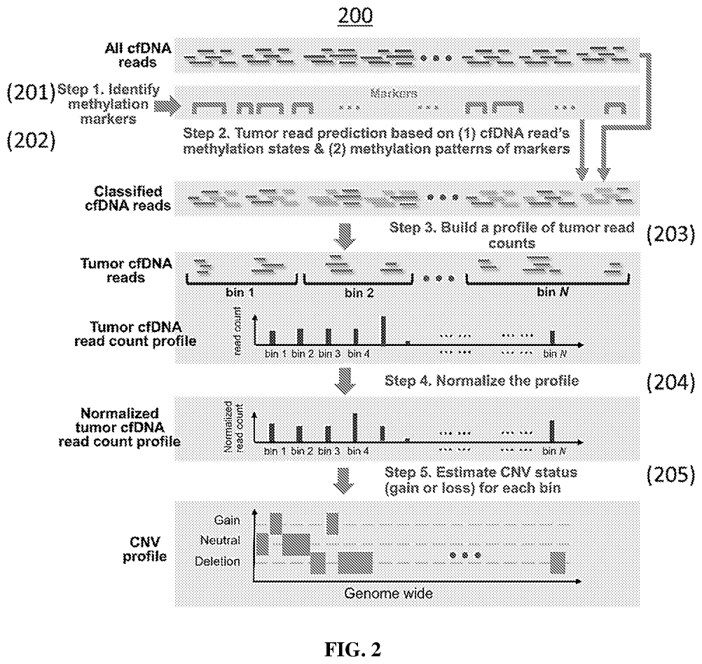

FIG. 2 illustrates examples of aspects of a method 200 for detecting CNVs in one or more cfDNA samples, according to a disclosed embodiment. The method 200 may comprise using cfDNA methylation sequencing data and cancer methylation markers to distinguish tumor-derived sequencing reads from normal sequencing reads. Each cfDNA sequencing read of a cfDNA sample may be classified as either corresponding to a tumor-derived cfDNA or a normal-plasma cfDNA, based on the methylation cfDNA sequencing data (e.g., obtained using a methylation sequencing method, such as Bisulfite sequencing) and cancer methylation markers. Based on this classification, only the set of tumor-derived sequencing reads may be utilized to infer CNV in a cfDNA sample. Accordingly, the method 200 may comprise identifying a set of cancer methylation markers (as in operation 201), predicting a set of tumor-derived sequencing reads (as in operation 202), constructing a profile of tumor-derived sequencing read counts across genomic bins (as in operation 203), normalizing the constructed profile across genomic bins (as in operation 204), and estimating CNV status for each genomic bin (as in operation 205). A diagnosis or prognosis may be made based on the inferred CNV profile of a subject. Alternatively, CNV inference approaches may have a wide range of applications, such as cancer monitoring, treatment monitoring, resistance monitoring, evaluation of efficacy of surgery or other treatment for a cancer of a subject, and minimal residual disease (MRD) detection. For example, minimum residual disease (MRD) may be detected using follow-up plasma cfDNA samples. That is, after surgery, a follow-up plasma sample can be obtained and analyzed using cfCNV methods and systems of the present disclosure to monitor and detect MRD. Because the tumor has been treated or resected, the tumor fraction in the follow-up cfDNA sample may be lower than in the baseline cfDNA sample. Therefore, MRD detection may require the sensitive and reliable detection of sequencing reads containing tumor-derived CNV signals provided by the methods and systems of the present disclosure.

### Cell-free Nucleic Acid Samples and Sequencing

The cell-free biological samples may be obtained or derived from a healthy subject, a patient with a disease or disorder (e.g., a cancer), a patient suspected of having a disease or disorder (e.g., a cancer), a pregnant female subject, or a female subject suspected of being pregnant. The cell-free samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a disease or disorder (e.g., a cancer), from a subject that is suspected of having a disease or disorder (e.g., a cancer), or from a subject that does not have or is not suspected of having the disease or disorder (e.g., a cancer).

The cell-free biological sample may be taken before and/or after treatment of a subject with the disease or disorder (e.g., a cancer). Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a disease or disorder (e.g., a cancer) for which a definitive positive or negative diagnosis is not available via clinical tests. The sample may be taken from a subject suspected of having a disease or disorder (e.g., a cancer). The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a disease or disorder (e.g., a cancer) due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, RNA-Seq (IIlumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. In some embodiments, the plurality of DNA is subjected to enzymatic or chemical reactions to distinguish methylated vs. unmethylated bases. In some embodiments, the plurality of DNA undergoes bisulfite conversion. Specific primers, possibly in conjunction with adapter ligation, may be used to selectively amplify certain targets for downstream sequencing. The PCR may comprise targeted amplification of one or more genomic loci, such as genomic loci associated with cancer or pregnancy. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers. The barcodes may uniquely tag the cfDNA molecules in a sample. Alternatively, the barcodes may non-uniquely tag the cfDNA molecules in a sample. The barcode(s) may non-uniquely tag the cfDNA molecules in a sample such that additional information taken from the cfDNA molecule (e.g., at least a portion of the endogenous sequence of the cfDNA molecule), taken in combination with the non-unique tag, may function as a unique identifier for (e.g., to uniquely identify against other molecules) the cfDNA molecule in a sample. For example, cfDNA sequence reads having unique identity (e.g., from a given template molecule) may be detected based on sequence information comprising one or more contiguous-base regions at one or both ends of the sequence read, the length of the sequence read, and the sequence of the attached barcodes at one or both ends of the sequence read. DNA molecules may be uniquely identified without tagging by partitioning a DNA (e.g., cfDNA) sample into many (e.g., at least about 50, at least about 100, at least about 500, at least about 1 thousand, at least about 5 thousand, at least about 10 thousand, at least about 50 thousand, or at least about 100 thousand) different discrete subunits (e.g., partitions, wells, or droplets) prior to amplification, such that amplified DNA molecules can be uniquely resolved and identified as originating from their respective individual input molecules of DNA.

The plurality of DNA molecule or derivatives may be subject to conditions sufficient to permit distinction between methylated nucleic acid bases and unmethylated nucleic acid bases. In some cases, subjecting the plurality of DNA molecules or derivatives thereof to conditions to distinguish methylated vs. unmethylated bases comprises performing bisulfite conversion on the plurality of DNA molecules. In some cases, subjecting the plurality of DNA molecules or derivatives thereof to conditions to distinguish methylated vs. unmethylated bases comprises enzymatic or chemical reactions to oxidize the methylated cytosine nucleic acid bases and/or hydroxymethylated cytosine nucleic acid bases followed by reduction and/or deamination of oxidation reaction products.

Samples of the present disclosure may be sequenced using various nucleic acid sequencing approaches. Such samples may be processed prior to sequencing, such as by being subjected to purification, isolation, enrichment, nucleic acid amplification (e.g., polymerase chain reaction (PCR)). Sequencing may be performed using, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing (e.g., Illumina, Pacific Biosciences of California, Ion Torrent), Single Molecule Sequencing by Synthesis (SMSS)(Helicos), massively-parallel sequencing, Clonal Single Molecule Array (Solexa), shotgun sequencing, Maxim-Gilbert sequencing, primer walking, sequencing using PacBio, SOLiD, Ion Torrent, or Nanopore platforms and any other sequencing methods known in the art. Simultaneous sequencing reactions may be performed using multiplex sequencing.

Sequencing may generate sequencing reads (“reads”), which may be processed by a computer. In some examples, reads may be processed against one or more references to identify copy number variants (CNVs).

In some examples, sequencing can be performed on cell-free polynucleotides that may comprise a variety of different types of nucleic acids. Nucleic acids may be polynucleotides or oligonucleotides. Nucleic acids included, but are not limited to deoxyribonucleic acid (DNA) or ribonucleic acid (RNA), single-stranded or double-stranded DNA, complementary DNA (cDNA), or a RNA/cDNA pair.

Identifying a Set of Cancer Methylation Markers that Covers the Genome

Pervasive hypomethylation in repeat regions is a hallmark of many cancer types. We therefore consider repeat sequences, which occupy more than 50% of the human genome, to identify a set of cancer methylation markers that sufficiently span the genome. As an example, for liver cancer, 447,050 markers were identified that had at least a change of average methylation level greater than 0.2 with respect to normal (note that the average methylation values span between 0 and 1). If the human genome is partitioned into 1 Mb bins, then each bin includes an average of 157 cancer markers, and 94% of all bins include cancer markers. These markers cover the entire genome. Therefore, we have a sufficient number of markers in each bin to construct a profile of tumor read fractions with high confidence.

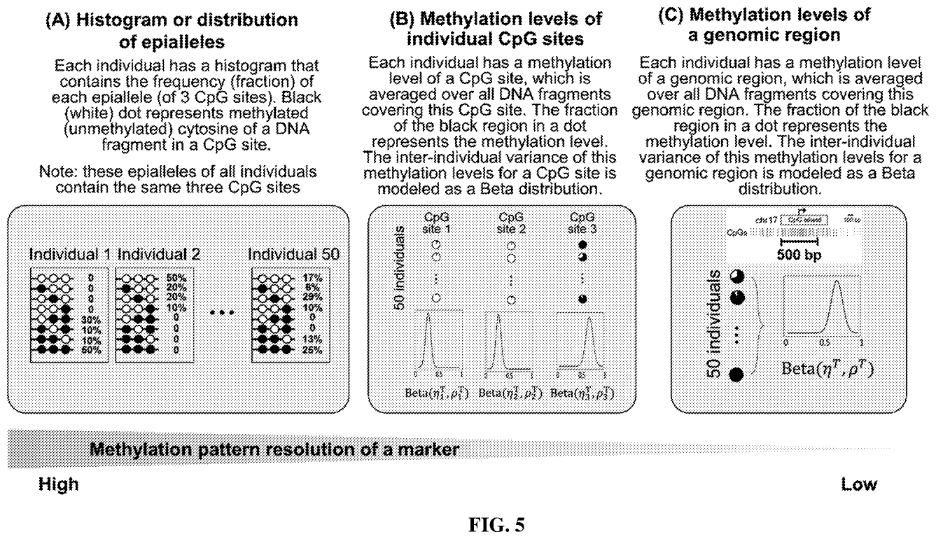

Referring to FIG. 2, in operation 201, there may be different methylation marker discovery methods that can be performed to identify cfDNA methylation markers. However, no matter which methylation marker discovery method is used, a key principle is to select a genomic region or an individual CpG site, whose methylation pattern can differentiate not only between tumors and their matched normal tissues (to remove tissue-specific effect), but also between tumors and normal plasma (to identify cancer-specific markers). The methylation pattern of a marker in either a tumor class or a normal class (normal tissues or normal cfDNA samples) can be defined at different base resolution levels. For example, as shown in FIG. 5, there may be three types of methylation patterns of a marker for a tumor class or normal class. Their resolution may be as high as epialleles, or may have a smaller base-resolution of “individual CpG sites,” or may be as low as the methylation level of a genomic region. For taking into account the inter-individual variance of a marker's methylation pattern in the population of a tumor (or normal) class, the statistical distribution, such as Beta distribution, of the marker can be used to describe the methylation pattern in a statistical manner. These distributions may be used in calculating class-specific likelihood of each sequencing read, as described herein.

### Predicting Tumor-Derived Sequencing Reads

For predicting cfDNA sequencing reads, methods and systems of the present disclosure may utilize the joint methylation patterns of a plurality of adjacent CpG sites on an individual cfDNA sequencing read. Conventional DNA methylation analysis may focus on the methylation rate of an individual CpG site in a cell population. This rate, often called the β-value of a CpG site, is the proportion of cells among a population of cells in which the given CpG site is methylated. However, approaches that use such population-average measures may not be sensitive enough to capture an abnormal methylation signal affecting only a small proportion of the cfDNAs.

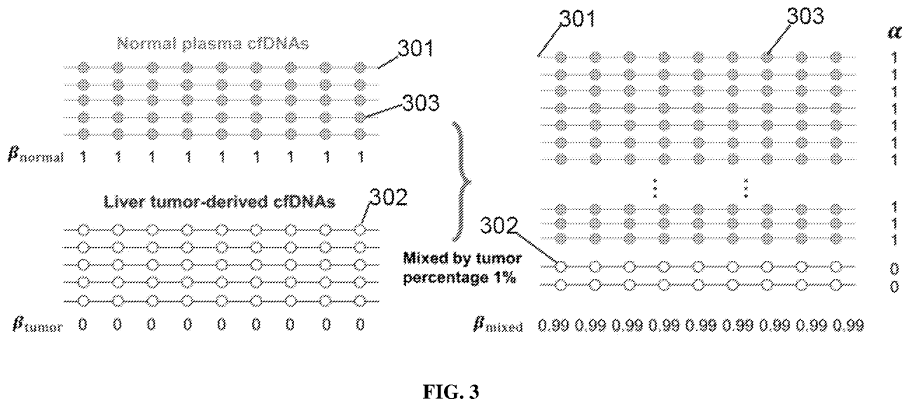

Referring to FIG. 3, the average methylation rates of the individual CpG sites may be βₙₒᵣₘₐₗ=1 for normal plasma cfDNAs, and βₜᵤₘₒᵣ=0 for tumor-derived cfDNAs; therefore, assuming the presence of about 1% tumor-derived cfDNAs among a cfDNA sample, the conventional measure yields a value of β_{mixed}=0.99 for a cfDNA sample (e.g., obtained from a subject having cancer), which may be difficult to differentiate from βₙₒᵣₘₐₗ=1 for a cfDNA sample (e.g., obtained from a subject not having cancer).

In contrast, methods and systems of the present disclosure may leverage the pervasive nature of DNA methylation to differentiate cancer-specific, tumor-derived cfDNA sequencing reads from normal cfDNA sequencing reads. If the methylation values of all of a plurality of CpG sites in a given sequencing read (denoted α-value) are averaged across the plurality of CpG sites, a striking difference may be observed between the abnormally methylated (e.g., tumor-derived) cfDNAs (αₜᵤₘₒᵣ=0%) and the normal (e.g., non-tumor-derived) cfDNAs (αₙₒᵣₘₐₗ=100%). As shown in FIG. 3, instead of averaging a plurality of observations of one CpG site vertically across all of a plurality of sequencing reads (β-value), systems and methods of the present disclosure may average observations across all of a plurality of CpG sites horizontally in a sequencing read (α-value). In other words, given the pervasive nature of DNA methylation, the joint methylation patterns of a plurality of adjacent CpG sites can be used to easily distinguish cancer-specific, tumor-derived cfDNA sequencing reads from normal cfDNA sequencing reads. As illustrated by the observations of α-value, tumor-specific signals arising from pervasive methylation in cfDNA may be effectively exploited to estimate whether the joint probability of all of a plurality of CpG sites in a given sequencing read is indicative of a DNA methylation signature of cancer. Using this probabilistic approach, systems and methods of the present disclosure may be effectively used to differentiate tumor-derived sequencing reads from normal sequencing reads.

FIG. 3 illustrates examples of concepts associated with distinguishing tumor-derived sequencing reads from normal sequencing reads in cfDNA, according to a disclosed embodiment. Each line 301 represents a sequencing read, and each dot represents a CpG site, where hollow dots 302 represent unmethylated CpG sites and solid dots 303 represent methylated CpG sites. Generally, tumor-derived sequencing reads may be expected to contain methylated CpG sites, while normal sequencing reads may be expected to contain unmethylated CpG sites. The α-value of a sequencing read (e.g., the observed methylation value averaged across all of a plurality of CpG sites in the given sequencing read, as shown by the vertical column) may be used to detect tumor-derived cfDNAs with a greater sensitivity, specificity, and accuracy than approaches that use the β-value of a CpG site (e.g., the observed methylation level of a CpG site averaged across all of a plurality of sequencing reads, as shown by the horizontal row), such as cases where the tumor-derived cfDNA fraction (e.g., among a cfDNA sample) is very low.

According to different embodiments, tumor-derived sequencing read prediction based on methylation patterns can be performed using a variety of different approaches. According to a preferred embodiment, tumor-derived sequencing read prediction based on methylation pattern is performed using either (1) the likelihood ratio or (2) the posterior probability, denoted by P(T|read). Both methods may comprising calculating the class-specific likelihoods of each cfDNA sequencing read, denoted by P(read|T) for the tumor class T and P(read|N) for the normal class N. For example, performing tumor read prediction is illustrated by operation 201 of FIG. 2.

To calculate the class-specific sequencing read likelihood, consider the tumor class T as an example, noting that a similar calculation can be applied to the normal class N. As motivated by the methylation measurement concept disclosed herein, P(read|T) can be calculated by assessing how well the joint methylation status of a plurality of CpG sites on the sequencing read fits the methylation pattern of class T. For example, the methylation pattern of a marker for class T can be obtained via biomarker discovery, which selects specific genomic regions that are able to differentiate between not only tumors and their matched normal tissues (for removing tissue-specific effect) but also between tumors and normal plasma (for identifying cancer-specific markers). A methylation pattern may describe the methylation levels of a plurality of adjacent CpG sites in a position-specific manner. A given CpG site may have methylation levels that exhibit inter-individual variance across a population of subjects. Therefore, the methylation levels of a given CpG site are commonly modeled as a Beta distribution with two positive shape parameters, Beta(η^{T},ρ^{T}). In addition, when the binary methylation status observed from sequencing data is considered, the Beta-Bernoulli distribution with the prior Beta(η^{T},ρ^{T}) has been demonstrated to be a more appropriate model.

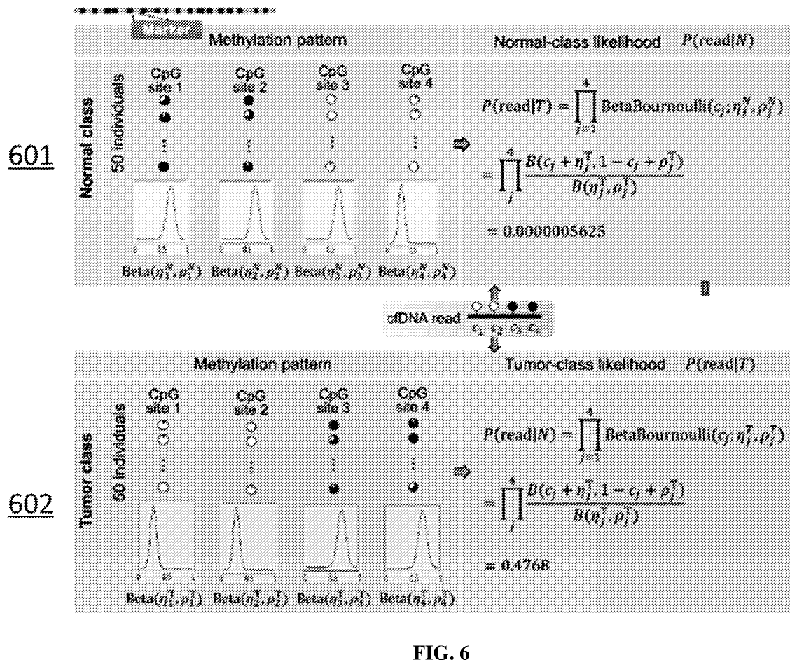

FIG. 6 illustrates an example of a method for calculating the class-specific likelihoods of a given cfDNA sequencing read, according to a disclosed embodiment, including a normal-class likelihood calculation 601 and a tumor-class likelihood calculation 602. The tumor-class likelihood calculation 602 illustrates an example of a tumor-specific methylation pattern, which contains a plurality of 4 CpG sites (CpG site 1, CpG site 2, CpG site 3, and CpG site 4) and has a statistical distribution of methylation levels for each of the CpG sites that is described by a Beta-Bernoulli distribution. The parameters of a Beta distribution, η^{T} and ρ^{T}, can be learned, for example, from the methylation data of solid tumors from a population of tumor patients (e.g., comprising 50 individuals). Therefore, given a cfDNA sequencing read containing this plurality of 4 CpG sites, methods and systems of the present disclosure may comprise calculating a likelihood of observing this sequencing read from the tumor class T (e.g., tumor class-specific sequencing read likelihood), denoted by P(read|T), as the probability of measuring how the joint-methylation-status of this sequencing read's plurality of 4 CpG sites simultaneously fits the 4 Beta-Bernoulli distributions of the tumor class. FIG. 6 illustrates details of the tumor-class likelihood calculation 602.

Similarly, the normal-class likelihood of the same sequencing read, denoted by P(read|N), can be computed, based on the marker's normal class methylation pattern. The normal-class likelihood calculation 601 illustrates an example of a normal methylation pattern, which contains a plurality of 4 CpG sites (CpG site 1, CpG site 2, CpG site 3, and CpG site 4) and has a statistical distribution of methylation levels for each of the CpG sites that is described by a Beta-Bernoulli distribution. The parameters of a Beta distribution, η^{N} and ρ^{N}, can be learned, for example, from the methylation data from a population (e.g., comprising 50 individuals) of normal subjects (e.g., not having cancer). Therefore, given a cfDNA sequencing read containing this plurality of 4 CpG sites, methods and systems of the present disclosure may comprise calculating a likelihood of observing this sequencing read from the normal class N (e.g., normal-class sequencing read likelihood), denoted by P(read|N), as the probability of measuring how the joint-methylation-status of this sequencing read's plurality of 4 CpG sites simultaneously fits the 4 Beta-Bernoulli distributions of the normal class. FIG. 6 illustrates details of the normal-class likelihood calculation 601.

In practice, a large amount of methylation data for tumor and matched tissue samples, such as those obtained from public data sources (e.g., The Cancer Genome Atlas (TCGA) database, the 1000 Genome database, and the International Cancer Genome Consortium database (ICGC)), may be profiled with Illumina bead arrays. Since the probes on the Illumina arrays may not cover all of a plurality of consecutive CpG sites in a CpG island, it may be impossible to specify the distribution of DNA methylation levels for individual CpG sites of the plurality in a marker. Therefore, in some embodiments, an “approximate” calculation of sequencing read likelihoods is used, based on an assumption that most CpG sites of the plurality within a marker region follow the same statistical distribution of methylation levels. In this manner, the methylation level of all of the plurality of CpG sites in a marker may be modeled by estimating a uniform Beta distribution. That is, each marker's methylation pattern for class T can be modeled as a Beta distribution, denoted by Beta(η^{T},ρ^{T}).

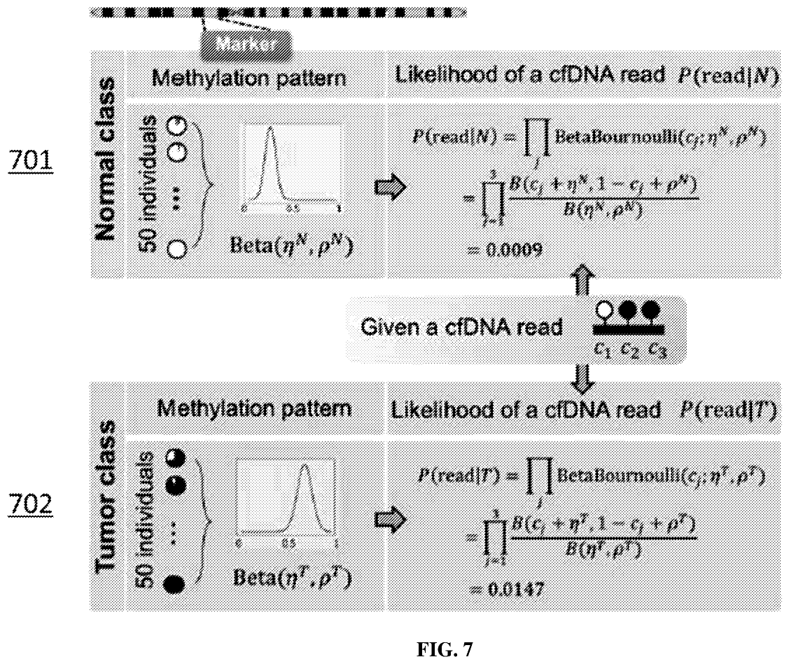

FIG. 7 illustrates an example of calculating a sequencing read's class-specific likelihoods, according to a disclosed embodiment, including a normal-class likelihood calculation 701 and a tumor-class likelihood calculation 702. According to the embodiment illustrated in FIG. 7, an assumption may be made that based on study results, the methylation of a plurality of CpG sites in a marker region, which covers less than 500 base pairs (bp), are highly correlated. For example, using a cohort of 711 normal samples comprising 18 tissue types collected from TCGA, the average correlation of adjacent CpG sites within each those markers was calculated to be 0.626 (P-values<10⁻³⁰).

The likelihood ratio method for classifying reads may be performed as follows. Based on the individual likelihoods of a sequencing read being derived from either a tumor class (T) or a normal tissue class (N), a likelihood ratio, denoted by Λ(r)=P(read|T)/P(read|N), may be calculated, which evaluates the relative likelihood (e.g., how many times more likely) that the sequencing read is derived from the tumor class T as compared to the normal tissue class N. The sequencing reads with large likelihood ratios (e.g., much larger than 1) are classified as tumor-derived sequencing reads. For example, a sequencing read may be classified as a tumor-derived sequencing read if its likelihood ratio is larger than a given likelihood ratio threshold (e.g., about 2, about 3, about 4, about 5, about 6, about 7, about 8, about 9, about 10, about 20, about 30, about 40, about 50, about 60, about 70, about 80, about 90, about 100, about 500, about 1000, about 5000, about 10⁴, about 5×10⁴, about 10⁵, about 5×10⁵, about 10⁶, about 5×10⁶, about 10⁷, about 5×10⁷, about 10⁸, about 5×10⁸, about 10⁹, or more than about 10⁹. In some embodiments, the p-value of each likelihood ratio may be calculated for evaluating its significance, and this p-value may be corrected in multiple testing. In some embodiments, different likelihood ratio (or p-value) thresholds may be applied to obtain multiple different sets of predicted tumor-derived sequencing reads with different qualities.

The posterior probability method for classifying reads may be performed as follows. The posterior probability, P(T|read), can be calculated based on Bayes theorem, using the following expression:

$$P ( T \vert read ) = \frac{θ P ( read \vert T )}{θ P ( read \vert T ) + ( 1 - θ ) P ( read \vert N )} ,$$

where θ is the tumor-derived cfDNA fraction. An optimization algorithm, such as an expectation maximization algorithm or a grid search algorithm, can be used to estimate θ by solving the following maximum likelihood estimation problem:

$${θ}^{*} = arg {max}_{θ} P ( R \vert θ ) Here ,$$

R={read₁, . . . , read_{N}} denotes the methylation sequencing data of a patient's cfDNAs, e.g., a set of N reads that are mapped to the genomic regions of all of a plurality of cancer methylation markers. The likelihood P(R|θ) can be expanded as the product of the likelihoods of all of the plurality of sequencing reads, e.g., P(R|θ)=Πᵢ₌₁^{N} P(readᵢ|θ). According to the mixture model, the likelihood P(readi|θ) of an individual read i may be given by a weighted sum of the class-specific sequencing read likelihoods, where the applied weights are the mixture parameter θ and (1−θ), as given by:

P(readᵢ|θ)=θP(readᵢ|T)+(1−θ)P(readᵢ|N)

The posterior probability may also be regarded as the quality score of a predicted tumor-derived sequencing read. In some embodiments, different thresholds of quality scores may be used to obtain multiple different sets of predicted tumor-derived sequencing reads, e.g., high-quality, medium-quality, and/or low-quality tumor-derived sequencing reads. Generally, sets of predicted tumor-derived sequencing reads obtained using larger thresholds of quality scores may be expected to be of higher quality as compared to sets of predicted tumor-derived sequencing reads obtained using smaller thresholds of quality scores. Among all optimization algorithms, the grid search algorithm may be used to find a global optimal value. It may be used to test all possible 10,000 values of θ uniformly distributed between 0% and 100%, and find the global optimal value with a precision of 0.01%, which is sufficient for capturing a tiny fraction of tumor-derived cfDNAs. Further, since the grid search is computationally fast, the estimate of θ can be easily refined by testing more precise values around the first optimal value. In some embodiments, in addition to or alternatively to the posterior probability method, sequencing reads may also be classified using the likelihood ratio.

As an alternative to the likelihood ratio and posterior probability methods for classifying sequencing reads, other methods may be applied to analyze methylation patterns of different classes (e.g., tumor-derived class or normal class) to classify sequencing reads. For example, such methylation pattern analysis may be based on epiallele patterns, such that a sequencing read can be classified as a tumor-derived sequencing read or a normal sequencing read based on whether or not its epiallele occurs more frequently in the tumor-derived class epiallele distribution or in the normal class epialleles distribution.

It should be appreciated that (1) methods and systems of the present disclosure may classify only sequencing reads that map to cancer markers with differential methylation patterns between tumor-derived sequencing reads and normal sequencing reads; and (2) due to the probabilistic nature of the calculations, some false positives (e.g., normal sequencing reads falsely predicted as tumor-derived sequencing reads) and false negatives (e.g., missed tumor-derived sequencing reads that are predicted as normal sequencing reads) may be generated that influence the CNV detection. However, approaches that use only tumor-derived sequencing reads with a minor fraction of false positives and/or false negatives may still be achieve higher accuracy, sensitivity, and/or specificity as compared to conventional approaches that use all sequencing reads (e.g., a mixture of tumor-derived sequencing reads and normal sequencing reads) of a cfDNA sample with a minor fraction of tumor-derived sequencing reads comparable in magnitude to the noise. Accordingly, utilizing methods and systems provided herein enables a significant enrichment of tumor-derived sequencing reads from the cfDNA sample. Further, as described in more detail herein, tumor read counts may be normalized in some embodiments in order to minimize the effect of false positives and/or false negatives.

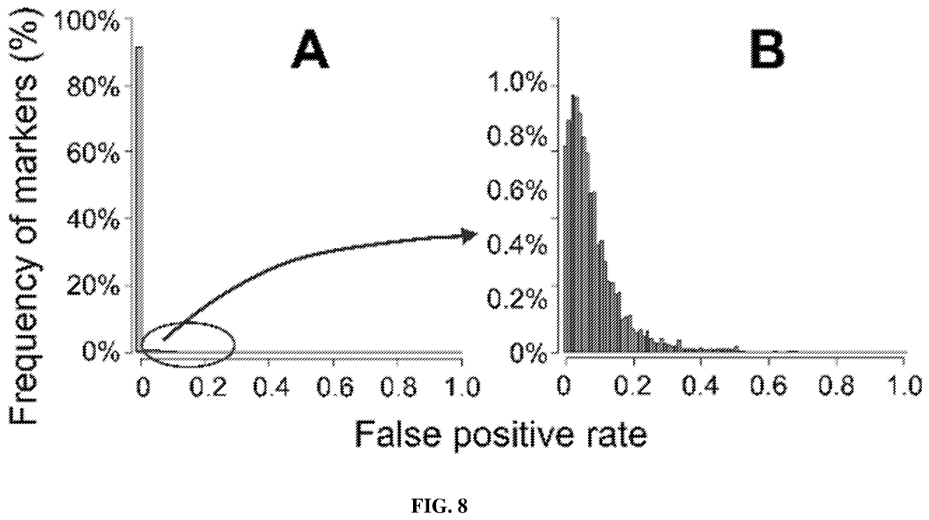

The classification accuracy of individual sequencing reads, which may be essential for CNV inference, may be assessed via various metrics of sequencing read classification, such as sensitivity, specificity, False Positive Rate (FPR), False Negative Rate (FNR), True Positive Rate (TPR), True Negative Rate (TNR), positive predictive value (PPV), negative predictive value (NPV), Area Under Curve (AUC), or a combination thereof. For example, FPR can be estimated by simply calling tumor-derived reads from plasma cfDNA of non-cancer individuals. The estimation of FNR may be more subtle, as the cancer markers used may be a superset of markers expected to present in any given subject's cfDNA sample, and hence may not all occur in a given cancer patient, and most tumor tissues are mixed with a substantial amount of normal tissues. FIG. 8 shows that the FPR rate from the cfDNA of a healthy individual may be extremely low for the vast majority of markers: about 90.9% of cancer markers have FPR of 0%, and about 8.3% of cancer markers have FPR below 20%. Such a low FPR rate, plus the ability of the normalized profile in leveraging all markers in a bin, may impact the CNV inference only in cases where the tumor fraction is extremely low.

### Constructing a Profile of Tumor-Derived Sequencing Read Counts

Referring to FIG. 2, in operation 202, a profile of the tumor-derived sequencing read counts is constructed. Based on the classification made in operation 201, a sequencing read count profile is constructed that excludes all sequencing reads classified as normal. Due to the challenge of low tumor-derived fraction in cfDNAs, in some embodiments, a genome-wide segmentation strategy may be applied by dividing the entire human genome into non-overlapping regions (bins) having a size of, for example, 1 M base pairs (bp). In some embodiments, the bins may have a size of about 100 bp, about 500 bp, about 1 kbp, about 5 kbp, about 10 kbp, about 50 kbp, about 100 kbp, about 500 kbp, about 1M bp, about 5M bp, about 10M bp, about 50M bp, about 100M bp, about 500M bp, or about 1000M bp. As such, in some embodiments, operation 202 comprises constructing a sequencing read count profile that excludes all sequencing reads among a plurality of sequencing reads that are classified as “normal.” Then, a genome-wide segmentation strategy may be adopted, comprising dividing the entire human genome into non-overlapping bins, where each bin may have a fixed size or a variable size.

Using a fixed bin size (e.g., of about 1M bp) may be advantageous for at least three reasons. First, large bins may be expected to include a sufficient number of tumor-derived sequencing reads, even at a shallow sequencing coverage. For instance, on average, a 1M bp bin includes 262 cancer markers, and 94% of all such bins are covered by cancer markers. Second, a bin size of 1M bp is large enough to overcome any biases related to nucleosome positioning, which is on the scale of about 166 bp and 332 bp. Third, it may be observed that this bin size works well on cfDNA data from actual samples.

It should be appreciated that different embodiments can utilize different bin sizes depending on, e.g., the tumor-derived sequencing read coverage. Also, the genome may be segmented into bins of varying size (e.g., automatically segmented using advanced segmentation methods). If tumor-derived sequencing reads may be identified using the likelihood ratio with a high quality score threshold, then the tumor-derived sequencing reads in each bin can be directed counted to create a high-quality profile. Alternatively, if tumor-derived sequencing reads are classified using the posterior probability, the sum of posterior probabilities over all of a plurality of sequencing reads within a bin may be calculated as the sequencing read count, as given by ΣᵢP(T|readᵢ). This method may work well because a sequencing read's posterior probability is a real value between 0 and 1, which is equivalent to a “fuzzy” representation of the sequencing read's identity.

Alternatively, a variable bin size may be used for a genome segmentation method that dynamically determines the optimal bin size based on sequencing depth and marker distribution. The genome may be dynamically segmented as follows. The marker regions in a bin may be required to contain a sufficient number of sequencing reads to ensure adequate sensitivity. Depending on the sequencing depth, it may be required that the total number of sequencing reads in each bin be above a threshold, in order to reach the sensitivity of detecting a small amount of tumor cfDNA. For example, if a detection sensitivity of 0.5% is desired, and at least 100 tumor reads per bin is required, then the bin must cover at least about 20,000 reads. A dynamic genome segmentation strategy may satisfy this criterion. First, the minimum total size of marker regions in each bin may be determined, according to the sequencing depth and the required sensitivity of cancer detection, satisfying the above criterion. Then, the whole genome may be divided into bins, such that each bin covers the determined size of marker regions, in order to satisfy the above first criterion. In some embodiments, since the CNV detection method relies on methylation markers, an alternative to dividing the genome into equally sized bins is to divide the genome into bins containing the same number or size of included marker regions. This criterion takes into account density variations in marker distribution across the genome.

### Normalizing the Constructed Profile

Again referring to FIG. 2, in operation 203, the constructed tumor-derived sequencing read profile is normalized. Marker's distribution, GC contents, sequencing read mapping, sequencing library construction, and sequencing depth and platforms can all introduce errors, biases, or noise in sequencing read counts. Normalizing the tumor-derived sequencing read profile may reduce such effects. In some embodiments, biases arising from GC content and capability may be corrected by using Locally Weighted Scatter-plot Smoothing (LOWESS) regression and various tools, such as HMMcopy. In addition, the bias correction may be improved by providing a control profile: in this context, generated from a matched normal sample comprising genomic DNA from white blood cells of the same blood sample from which the cfDNA sample was obtained (white blood cells usually contribute ˜80% cfDNA). If no white blood cell sample of the same patient is available, it may be substituted with a control reference data set (e.g., constructed from a collection of cfDNA samples from healthy subjects). More importantly, comparing a constructed tumor-derived sequencing read profile with the control profile may also reduce the false-positive sequencing reads in the case profile that are caused by low-quality cancer markers. As another example, another approach for bias correction is within-sample tumor-derived sequencing read profile comparison, in which, the reference profile is constructed from certain genomic regions within the same sample. Finally, the log ratios between case and control samples for each bin may then be used as the normalized profile. In addition to the above described method, the “local” tumor cfDNA fraction of each bin (θ_{bin}) may be used as a normalized measure of tumor read abundance in a bin. Specifically, the “local” tumor fraction Obin for a single bin is the fraction of tumor-derived sequencing reads among all of the plurality of sequencing reads that are mapped to the markers within the bin, and can be estimated by applying a maximum likelihood estimation method, as described herein, to all of the plurality of sequencing reads that are mapped to the markers within a single bin.

### Estimating CNV Status (Gain or Loss)

Again referring to FIG. 2, in operation 204, a CNV status (e.g., gain or loss) of each genomic region is inferred. This is performed for each bin, from which a cancer diagnosis or prognosis may be made for a subject. After normalization, the sequencing read count data may be conceptually similar to the probe log ratios from arrayCGH data. Therefore, algorithms to detect CNV regions from arrayCGH data, such as CBS and CGHseg, can be reused and modified to be applied to sequencing read count data. In view of the foregoing, in some embodiments, operation 204 comprises utilizing the normalized profile output for estimating the CNV status. Various suitable algorithms to detect CNV regions can be used to analyze this normalized profile.

### Performing a Diagnosis Based on CNV Inference

After the CNV status of the genomic regions are inferred, a diagnosis or prognosis may be determined based on the foregoing inferences. In order to determine a diagnosis decision, e.g., “whether the patient has cancer,” the fraction of bins with an abnormal sequencing read count (e.g., based on log-ratios) may be used as a cancer indicator score, by way of example. In other words, in some embodiments, the diagnosis or prognosis is determined based upon the fraction of bins with abnormal sequencing read count (log-ratios) as a cancer indicator score. As another example, the cancer indicator score can be determined by the occurrence of gains or losses in recurrent chromosome regions, such as losses at the APC gene region for colon cancer.

This approach may be found to achieve good diagnosis results. In various embodiments, steps 201-204 may include certain variations and/or sub-operations that are within the scope of the methods and systems of the present disclosure.

As discussed, FIG. 6 illustrates an example of a method for calculating the class-specific likelihoods of a given cfDNA sequencing read with a plurality of 4 CpG sites (e.g., c₁c₂c₃c₄=0011), where “0011” denotes that the first two CpG sites of the plurality are unmethylated and the last two CpG sites of the plurality are methylated. Note that (1) the binary methylation status of each CpG site can be modeled as a Beta-Bernoulli distribution with prior Beta(η,ρ), denoted by cⱼ˜BetaBournoulli (ηⱼ,ρⱼ), so the likelihood of observing methylation status cⱼ in CpG site j can be represented as BetaBournoulli (cⱼ,ηⱼ,ρⱼ); and (2) B(x,y) is the beta function.

As also discussed, FIG. 7 illustrates an example of a method for “approximately” calculating the class-specific likelihoods of a given cfDNA sequencing read, when the methylation patterns of tumor and normal classes follow Beta distributions Beta(η^{T},ρ^{T}) and Beta(η^{N},ρ^{N}), respectively. Note that B(x,y) is the beta function.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and therefore can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Application of cfCNV Methods to Liver Cancer Samples to Deconvolve Tumor cfDNA and Detect Cancer

A cfCNV method was implemented as follows. In operations 1 and 2, the posterior probability method was utilized to classify and count tumor-derived sequencing reads from among a plurality of sequencing reads obtained from cfDNA samples of liver cancer patients. In step 3 only white blood cells from the same blood sample were utilized to construct a control profile for normalization, without considering other sources of experimental and technical bias. In step 4, the fraction of bins with abnormally log-ratios was utilized as the final cancer indicator score.

To perform an example of a method according to disclosed embodiments, whole genome bisulfite sequencing (WGBS) data of plasma cfDNA samples were collected from 15 liver cancer patients and 5 healthy subjects.

The performance of a cfCNV method was compared to that of a conventional sequencing read-count (RC) method. For differentiating tumor-derived sequencing reads, methylation markers, most of which are located in gene promoter regions, and hypomethylation markers in repeat regions were used. Using these samples, it was demonstrated that cfCNV methods are more sensitive and accurate for detecting cancer than the conventional read-count method.

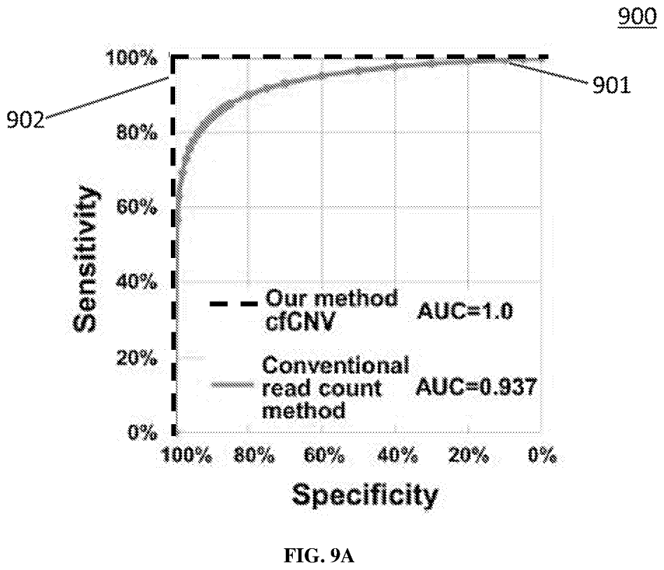

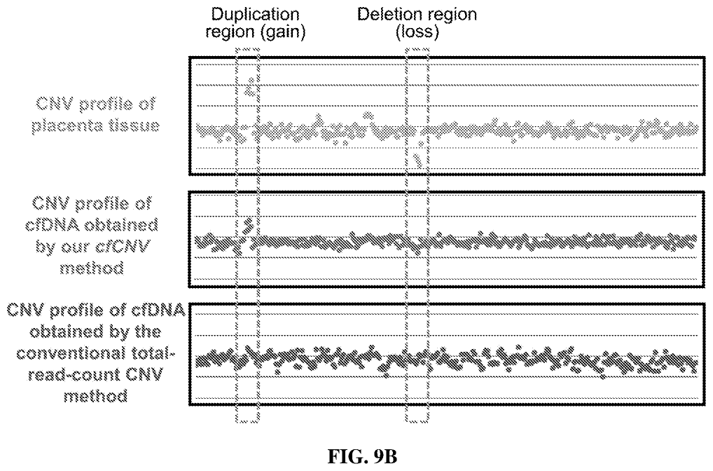

Specifically, as shown in FIG. 9A, referring to chart 900, a disclosed embodiment of the cfCNV method achieved a sensitivity of 100% with a specificity of 100% (with the area under curve of the ROC (AUC) of 1.0, where the ROC was generated using different cutoffs of the cancer indicator score for diagnosis). This ROC curve is shown by solid line 902. In contrast, the conventional read-count method (with a ROC curve shown by dashed line 901) achieved a sensitivity of 62.8% with a specificity of 99% (with an area under curve of the ROC (AUC) of 0.937). In addition, how well the CNV-based cancer indicator scores derived from both methods correlate with tumor size was assessed. Among all 15 liver cancer patients with tumor size records, the cancer indicator score (e.g., fraction of abnormal CNV bins) achieved a Pearson's correlation of 0.881. In comparison, the same cancer indicator used in the conventional read-count method achieved a Pearson's correlation of 0.700.

It should be appreciated that embodiments described herein are envisioned as being modified in different ways. For example, in detecting small CNVs, using a bin size of 1M base pairs ensures a sufficient number of tumor-derived sequencing reads for CNV detection, but flattens the signals of small CNVs. Therefore, an embodiment may comprise adapting advanced genome segmentation methods to automatically identify CNV bins that have variable size. Further, correction of systematic biases by the simultaneous analysis of multiple cfDNA samples may be improved. Some potential systematic biases that cannot be identified in a single sample, such as poor marker qualities, may be easily identified by modelling sequencing read counts across multiple samples in each genomic region. Such a population-based strategy may fully utilize the information of multiple cfDNA samples, and may be shown to achieve better CNV detection performance than using only a single sample.

### Example 2

### Further Improvements on cfCNV Methods

The cfCNV methods described herein may be improved by one or more of the following approaches.

First, the cfCNV methods may detect small CNVs. Generally, using a bin size of 1M base pairs ensures a sufficient number of tumor-derived sequencing reads for CNV detection, but flattens the signals of small CNVs. Therefore, advanced genome segmentation methods are adapted to automatically identify CNV bins that have variable size.

Second, the cfCNV methods may improve the correction of systematic biases by the simultaneous analysis of multiple cfDNA samples. Some potential systematic biases that cannot be identified in a single sample, such as poor-quality markers, are easily identified by modelling sequencing read counts across multiple samples in each genomic region. Such a population-based strategy can fully utilize the information of multiple cfDNA samples, and achieves higher-performance CNV detection as compared to using only a single sample. The strategies used in JointSLM23 framework or principal-component analysis (as used in XHMM24) are adapted to integrate multiple samples for bias removal.

Third, the cfCNV methods may account for sequencing error and/or bisulfite conversion rates as follows. Generally, sequencing errors and/or incomplete bisulfite conversion may impact the likelihood estimates P(read|T) and P(read|N). The sequencing error of a CpG site can be calculated using the base quality and read mapping quality scores. The incomplete bisulfite conversion rate is not site-dependent and may be estimated from cytosines that are known to be unmethylated (e.g., the mitochondrial genome). The distribution of joint methylations among multiple adjacent CpG sites may be estimated, while taking into account either or both of these factors.

### Example 3

### Detecting Prenatal Conditions by Inferring CNVs of Placental/Fetal DNA

The methods and systems described herein may be used to infer placental CNVs for detecting prenatal conditions (e.g., diseases or disorders of a pregnant subject or of a fetus of a pregnant subject) via methylation sequencing data analysis of maternal cfDNA. Specifically, particular genomic regions or individual CpG sites, whose methylation patterns (see FIG. 5 for three kinds of patterns at different resolutions) can differentiate placenta from all other normal tissues and normal cfDNA samples, were selected as fetal methylation markers. Other steps of the analysis remain the same (as for the detection of CNV in cancer), other than using the plurality of placenta methylation markers (instead of cancer markers). A profile of normalized placenta read abundance is constructed and used for estimating CNV status in each genomic bin. The inferred CNV status is then used for detecting prenatal conditions, such as a fetal aneuploidy (e.g., Down syndrome).

To simulate CNVs in the placenta sample, CNV gain and loss were simulated in the placenta sample as follows: 50% of reads in the region of size 40 M base pairs (bp) in the genome were duplicated to construct a duplication region, and 50% of reads in another region of size 40 M base pairs (bp) were removed to construct a deletion region. The methylation data of a plasma cfDNA sample was simulated by sampling and mixing the methylation sequencing reads of two samples, a normal plasma cfDNA sample and a solid placenta sample. The solid placenta sample has simulated CNVs (as described elsewhere herein). Simulated plasma cfDNA samples were generated with placenta fractions of 10%, 5%, and 3%.

A variable-bin genome segmentation method was implemented to define the variable-sized bins. Tissue deconvolution was performed to predict placenta reads, and then the CNV profile was constructed based on these bins. To evaluate the performance of a variable-sized genome segmentation method and a cfCNV method of the present disclosure, a comparison was performed between the CNV profiles of the solid placenta tissue in the pregnant subject (regarded as the true CNV) and the CNV profiles of the simulated cfDNA samples of the same subject, which can be either obtained by the cfCNV method, or by a traditional total-read-count-based CNV method. The comparison can be performed by calculating the correlation of the solid placenta tissue's CNV profile and the cfDNA-derived CNV profile.

Table 1 illustrates examples of aspects of results achieved by a cfCNV method, according to a disclosed embodiment. Given a set of simulated cfDNA samples of pregnant subjects at different placenta fractions of 10%, 5%, and 3%, the cfCNV method can construct a CNV profile that matches well with the CNV profile of the solid placenta tissue. As shown in Table 1, the cfDNA CNV profile obtained by the cfCNV method has a much higher correlation with the solid placenta tissue's CNV profile, as compared to that obtained by a traditional total-read-count-based CNV method. Note that total-read-count-based CNV methods are commonly used in conventional methods of counting the total sequencing reads in a bin and to perform normalization of the total read counts. These results demonstrate that the cfCNV method can improve performance of CNV profiling.

FIG. 9B illustrates examples of aspects of results achieved by a disclosed embodiment. This figure further demonstrates that the cfCNV method can sensitively detect the same duplication regions (e.g., indicative of CNV gain) and deletion regions (e.g. indicative of CNV loss) as those found in a solid placenta tissue sample from the same subject. In comparison, the traditional CNV method (e.g., total read count-based CNV method) fails to do so.

TABLE 1
Comparisons of correlation between a CNV profile of a placenta
tissue sample and CNV profiles of simulated cfDNA samples obtained by a cfCNV
method of the present disclosure and by a conventional read count-based CNV method.

| | Correlation between placenta | | Correlation between | |
|---|---|---|---|---|
| | tissue's CNV profile, and | | placenta tissue's CNV | |
| | cfDNA's CNV profile of the | | profile, and cfDNA's CNV | |
| Simulated | same subject obtained by | | profile of the same subject | |
| placenta | conventional read count-based | | obtained by a cfCNV method | |
| fraction | CNV method | P value | of the present disclosure | P value |
| 10% | 0.337 | 8.21E−11 | 0.865 | 0 |
| 5% | 0.251 | 1.73E−06 | 0.689 | 0 |
| 3% | 0.217 | 3.93E−05 | 0.494 | 0 |

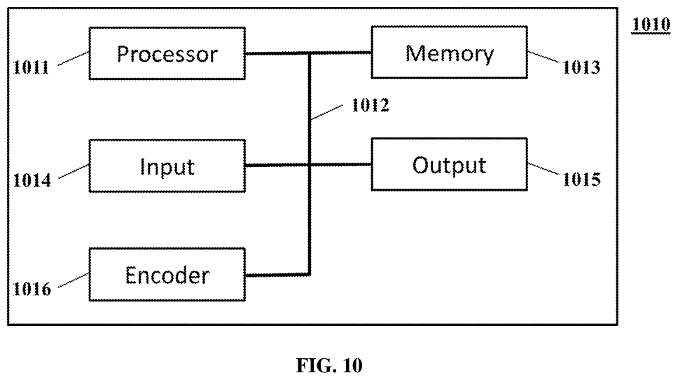

FIG. 10 shows an exemplary system adapted to sensitively detect CNVs from cell-free nucleic acid, such as cell-free deoxyribonucleic acid (cfDNA) and cell-free ribonucleic acid (cfRNA), in accordance with the present disclosure. Electronic device 1010 can comprise various configurations of devices. For example, electronic device 1010 can comprise a computer, a laptop computer, a tablet device, a server, a dedicated spatial processing component or device, a smartphone, a personal digital assistant (PDA), an Internet of Things (IOTA) device, a network equipment (e.g., router, access point, femtocell, Pico cell, etc.), and/or the like.

Electronic device 1010 can comprise any number of components operable to facilitate functionality of electronic device 1010 in accordance with the present disclosure, such as processor(s) 1011, system bus 1012, memory 1013, input interface 1014, output interface 1015, and encoder 1016 of the illustrated embodiment. Processor(s) 1011 can comprise one or more processing units, such as a central processing unit (CPU) (e.g., a processor from the Intel CORE family of multi-processor units), a field programmable gate array (FPGA), and/or an application specific integrated circuit (ASIC), operable under control of one or more instruction sets defining logic modules configured to provide operation as described herein. System bus 1012 couples various system components, such as memory 1013, input interface 1014, output interface 1015 and/or encoder 1016 to processor(s) 1011. Accordingly, system bus 1012 of embodiments may be any of various types of bus structures, such as a memory bus or memory controller, a peripheral bus, and/or a local bus using any of a variety of bus architectures. Additionally or alternatively, other interface and bus structures, such as a parallel port, game port or a universal serial bus (USB) may be utilized. Memory 1013 can comprise various configurations of volatile and/or nonvolatile computer-readable storage media, such as RAM, ROM, EPSOM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Input interface 1014 facilitates coupling one or more input components or devices to processor(s) 1011.

For example, a user may enter commands and information into electronic device 1010 through one or more input devices (e.g., a keypad, microphone, digital pointing device, touch screen, etc.) coupled to input interface 1014. Image capture devices, such as a camera, scanner, 3-D imaging device, etc., may be coupled to input interface 1014 of embodiments, such as to provide source video herein. Output interface 1015 facilitates coupling one or more output components or devices to processor(s) 1011. For example, a user may be provided output of data, images, video, sound, etc. from electronic device 1010 through one or more output devices (e.g., a display monitor, a touch screen, a printer, a speaker, etc.) coupled to output interface 1015. Output interface 1015 of embodiments may provide an interface to other electronic components, devices and/or systems (e.g., a memory, a video decoder, a radio transmitter, a network interface card, devices such as a computer, a laptop computer, a tablet device, a server, a dedicated spatial processing component or device, a smartphone, a PDA, an IOTA device, a network equipment, a set-top-box, a cable headend system, a smart TV, etc.).

### Computer Systems

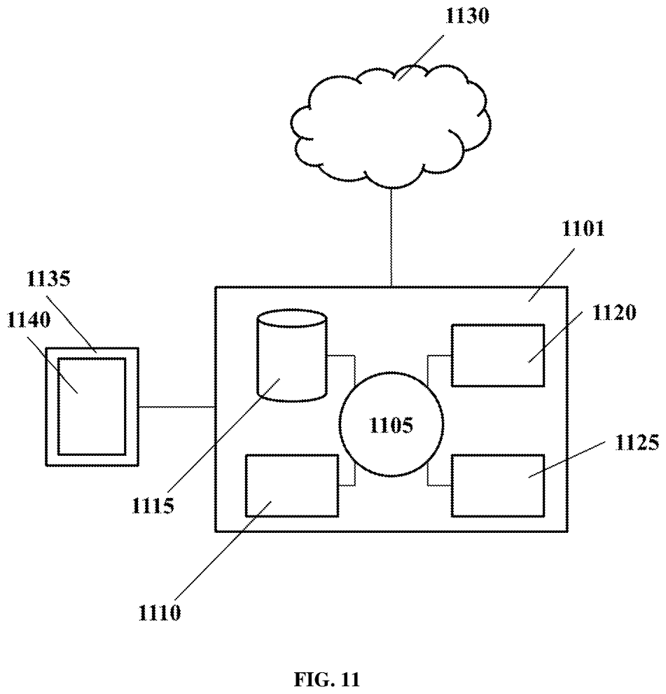

The present disclosure provides computer systems that are programmed to implement methods of the disclosure. FIG. 11 shows a computer system 1101 that is programmed or otherwise configured to, for example, obtain a plurality of sequencing reads; sequence a plurality of cell-free nucleic acids; classify sequencing reads as a tumor-derived sequencing read or a normal sequencing read; construct a profile of tumor-derived sequencing read counts; normalize a constructed profile of tumor-derived sequencing read counts; infer a CNV status for each of a plurality of genomic regions; calculate a likelihood ratio for a sequencing read; calculate a posterior probability for a sequencing read; calculate a class-specific likelihood for a sequencing read; perform a bias correction of a constructed profile; detect a cancer of a subject based on inferred CNV statuses; classify sequencing reads as a fetal-derived sequencing read or a normal sequencing read; construct a profile of fetal-derived sequencing read counts; normalize a constructed profile of fetal-derived sequencing read counts; and detect a fetal anomaly of a fetus of a pregnant subject based on inferred CNV statuses.

The computer system 1101 can regulate various aspects of analysis, calculation, and generation of the present disclosure, such as, for example, obtaining a plurality of sequencing reads; sequencing a plurality of cell-free nucleic acids; classifying sequencing reads as a tumor-derived sequencing read or a normal sequencing read; constructing a profile of tumor-derived sequencing read counts; normalizing a constructed profile of tumor-derived sequencing read counts; inferring a CNV status for each of a plurality of genomic regions; calculating a likelihood ratio for a sequencing read; calculating a posterior probability for a sequencing read; calculating a class-specific likelihood for a sequencing read; performing a bias correction of a constructed profile; detecting a cancer of a subject based on inferred CNV statuses; classifying sequencing reads as a fetal-derived sequencing read or a normal sequencing read; constructing a profile of fetal-derived sequencing read counts; normalizing a constructed profile of fetal-derived sequencing read counts; and detecting a fetal anomaly of a fetus of a pregnant subject based on inferred CNV statuses. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet.

The network 1130 in some cases is a telecommunication and/or data network. The network 1130 can include one or more computer servers, which can enable distributed computing, such as cloud computing. For example, one or more computer servers may enable cloud computing over the network 1130 (“the cloud”) to perform various aspects of analysis, calculation, and generation of the present disclosure, such as, for example, obtaining a plurality of sequencing reads; sequencing a plurality of cell-free nucleic acids; classifying sequencing reads as a tumor-derived sequencing read or a normal sequencing read; constructing a profile of tumor-derived sequencing read counts; normalizing a constructed profile of tumor-derived sequencing read counts; inferring a CNV status for each of a plurality of genomic regions; calculating a likelihood ratio for a sequencing read; calculating a posterior probability for a sequencing read; calculating a class-specific likelihood for a sequencing read; performing a bias correction of a constructed profile; detecting a cancer of a subject based on inferred CNV statuses; classifying sequencing reads as a fetal-derived sequencing read or a normal sequencing read; constructing a profile of fetal-derived sequencing read counts; normalizing a constructed profile of fetal-derived sequencing read counts; and detecting a fetal anomaly of a fetus of a pregnant subject based on inferred CNV statuses. Such cloud computing may be provided by cloud computing platforms such as, for example, Amazon Web Services (AWS), Microsoft Azure, Google Cloud Platform, and IBM cloud. The network 1130, in some cases with the aid of the computer system 1101, can implement a peer-to-peer network, which may enable devices coupled to the computer system 1101 to behave as a client or a server.

The CPU 1105 may comprise one or more computer processors and/or one or more graphics processing units (GPUs). The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, a visual display of data indicative of sequencing reads, methylation sequencing data, tumor-derived sequencing reads, normal sequencing reads, a profile of tumor-derived sequencing read counts, inferred CNV statuses, and/or a detected cancer of a subject; and an identification of a subject as having a cancer. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, obtain a plurality of sequencing reads; sequence a plurality of cell-free nucleic acids; classify sequencing reads as a tumor-derived sequencing read or a normal sequencing read; construct a profile of tumor-derived sequencing read counts; normalize a constructed profile of tumor-derived sequencing read counts; infer a CNV status for each of a plurality of genomic regions; calculate a likelihood ratio for a sequencing read; calculate a posterior probability for a sequencing read; calculate a class-specific likelihood for a sequencing read; perform a bias correction of a constructed profile; detect a cancer of a subject based on inferred CNV statuses; classify sequencing reads as a fetal-derived sequencing read or a normal sequencing read; construct a profile of fetal-derived sequencing read counts; normalize a constructed profile of fetal-derived sequencing read counts; and detect a fetal anomaly of a fetus of a pregnant subject based on inferred CNV statuses.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Moreover, the scope of the present application is not intended to be limited to the particular embodiments of the process, machine, manufacture, composition of matter, means, methods and steps described in the specification.

1. A method for detecting copy number variants (CNVs) from a plurality of cell-free nucleic acids of a subject, the method comprising:
obtaining a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and
using methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises:
classifying a sequencing read of the methylation sequencing data as a tumor-derived sequencing read or a normal sequencing read;
constructing a profile of tumor-derived sequencing read counts, wherein constructing the profile comprises quantifying the plurality of tumor-derived sequencing reads at each of a plurality of genomic regions;
normalizing the constructed profile of tumor-derived sequencing read counts, to produce a normalized profile of tumor-derived sequencing read counts; and
inferring a CNV status for each of the plurality of genomic regions based on the normalized profile of tumor-derived sequencing read counts.

2. The method of claim 1, wherein classifying a sequencing read of the methylation sequencing data as a tumor-derived sequencing read or a normal sequencing read comprises at least one of:
(i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and
(ii) calculating a posterior probability for the sequencing read, and comparing the posterior probability to a posterior probability threshold, wherein a posterior probability that exceeds the posterior probability threshold indicates a tumor-derived sequencing read.

3. The method of claim 2, wherein classifying the sequencing read as a tumor-derived sequencing read or a normal sequencing read further comprises:
calculating a class-specific likelihood for the sequencing read.

4. The method of any of claims 1-3, wherein constructing the profile of tumor-derived sequencing read counts comprises excluding all of the plurality of sequencing reads classified as a normal sequencing read.

5. The method of any of claims 1-3, wherein constructing the profile of tumor-derived sequencing read counts comprises dividing at least a portion of the human genome into the plurality of genomic regions, the plurality of genomic regions comprising non-overlapping bins, according to a genome-wide segmentation strategy.

6. The method of claim 5, wherein the non-overlapping bins have a fixed size.

7. The method of claim 5, wherein the non-overlapping bins vary in size.

8. The method of any of claims 1-7, wherein normalizing the constructed profile of the tumor-derived sequencing read counts comprises calculating a fraction of tumor-derived cell-free nucleic acids in each of the plurality of genomic regions of the constructed profile.

9. The method of any of claims 1-7, wherein normalizing the constructed profile of the tumor-derived sequencing read counts comprises performing a bias correction of the constructed profile.

10. The method of claim 9, wherein performing the bias correction reduces bias attributable to at least one of: GC contents, sequencing read mapping, sequencing library construction, and sequencing platforms.

11. The method of claim 9, wherein performing the bias correction comprises comparing the constructed profile to a reference profile.

12. The method of claim 11, wherein the reference profile is a matched normal sample comprising genomic DNA from white blood cells obtained from a same blood sample as the plurality of cell-free nucleic acids.

13. The method of claim 11, wherein the reference profile is constructed from one or more cfDNA samples obtained from healthy subjects.

14. The method of claim 11, wherein the reference profile is constructed from certain genomic regions within a same sample.

15. The method of any of claims 1-14, wherein normalizing the constructed profile of tumor-derived sequencing read counts comprises measuring log ratios between case and control samples for each of the plurality of genomic regions.

16. The method of any of claims 1-15, further comprising detecting a cancer of the subject based on the plurality of inferred CNV statuses.

17. The method of claim 16, wherein the cancer is detected based on a fraction of one or more genomic regions having tumor-derived sequencing read counts, and wherein the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

18. The method of any of claims 1-17, further comprising using the CNV status for treatment monitoring of the subject.

19. The method of any of claims 1-18, further comprising using the CNV status for patient stratification of the subject.

20. The method of any of claims 1-19, further comprising using CNV status for tracing a tissue-of-origin of the plurality of cell-free nucleic acids.

21. The method of any of claims 1-20, further comprising identifying the at least one cancer methylation marker by processing methylation data of solid tumor samples, normal tissue samples, cell-free nucleic acid samples, or a combination thereof, obtained from one or more additional subjects.

22. The method of claim 21, wherein the at least one cancer methylation marker comprises epialleles, individual CpG sites, genomic regions, or a combination thereof.

23. The method of claim 21, wherein processing the methylation data comprises identifying the at least one cancer methylation marker based on a differential methylation of the at least one cancer methylation marker between the solid tumor samples, the normal tissue samples, the cell-free nucleic acid samples, or the combination thereof.

24. The method of claim 21, wherein the one or more additional subjects comprise one or more cancer patients and one or more normal subjects.

25. The method of claim 24, wherein processing the methylation data comprises identifying the at least one cancer methylation marker based on a differential methylation of the at least one cancer methylation marker between samples obtained from the one or more cancer patients and samples obtained from the one or more normal subjects.

26. A system for detecting copy number variants (CNVs) from a plurality of cell-free nucleic acids of a subject, the system comprising:
a memory;
one or more processors communicatively coupled to the memory, the one or more processors individually or collectively programmed to:
obtain a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and
use methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises:
classifying a sequencing read of the methylation sequencing data as a tumor-derived sequencing read or a normal sequencing read;
constructing a profile of tumor-derived sequencing read counts, wherein constructing the profile comprises quantifying the plurality of tumor-derived sequencing reads at each of a plurality of genomic regions;
normalizing the constructed profile of tumor-derived sequencing read counts, to produce a normalized profile of tumor-derived sequencing read counts; and
inferring a CNV status for each of the plurality of genomic regions based on the normalized profile of tumor-derived sequencing read counts.

27. The system of claim 26, wherein classifying a sequencing read of the methylation sequencing data as a tumor-derived sequencing read or a normal sequencing read comprises at least one of:
(i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and
(ii) calculating a posterior probability for the sequencing read, and comparing the posterior probability to a posterior probability threshold, wherein a posterior probability that exceeds the posterior probability threshold indicates a tumor-derived sequencing read.

28. The system of claim 27, wherein classifying the sequencing read as a tumor-derived sequencing read or a normal sequencing read further comprises:
calculating a class-specific likelihood for the sequencing read.

29. The system of any of claims 26-28, wherein constructing the profile of tumor-derived sequencing read counts comprises excluding all of the plurality of sequencing reads classified as a normal sequencing read.

30. The system of any of claim 26-28, wherein constructing the profile of tumor-derived sequencing read counts comprises dividing at least a portion of the human genome into the plurality of genomic regions, the plurality of genomic regions comprising non-overlapping bins, according to a genome-wide segmentation strategy.

31. The system of claim 30, wherein the non-overlapping bins have a fixed size.

32. The system of claim 30, wherein the non-overlapping bins vary in size.

33. The system of any of claims 26-32, wherein normalizing the constructed profile of the tumor-derived sequencing read counts comprises calculating a fraction of tumor-derived cell-free nucleic acids in each of the plurality of genomic regions of the constructed profile.

34. The system of any of claims 26-32, wherein normalizing the constructed profile of the tumor-derived sequencing read counts comprises performing a bias correction of the constructed profile.

35. The system of claim 34, wherein performing the bias correction reduces bias attributable to at least one of: GC contents, sequencing read mapping, sequencing library construction, and sequencing platforms.

36. The system of claim 34, wherein performing the bias correction comprises comparing the constructed profile to a reference profile.

37. The system of claim 36, wherein the reference profile is a matched normal sample comprising genomic DNA from white blood cells obtained from a same blood sample as the plurality of cell-free nucleic acids.

38. The system of claim 36, wherein the reference profile is constructed from one or more cfDNA samples obtained from healthy subjects.

39. The method of claim 36, wherein the reference profile is constructed from certain genomic regions within a same sample.

40. The system of any of claims 26-39, wherein normalizing the constructed profile of tumor-derived sequencing read counts comprises measuring log ratios between case and control samples for each of the plurality of genomic regions.

41. The system of any of claims 26-40, wherein the one or more processors are programmed to detect a cancer of the subject based on the plurality of inferred CNV statuses.

42. The system of claim 41, wherein the cancer is detected based on a fraction of one or more genomic regions having tumor-derived sequencing read counts, and wherein the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

43. The system of any of claims 26-42, wherein the one or more processors are individually or collectively programmed to further use the CNV status for treatment monitoring of the subject.

44. The system of any of claims 26-43, wherein the one or more processors are individually or collectively programmed to further use the CNV status for patient stratification of the subject.

45. The system of any of claims 26-44, wherein the one or more processors are individually or collectively programmed to further use the CNV status for tracing a tissue-of-origin of the plurality of cell-free nucleic acids.

46. The system of any of claims 26-45, wherein the one or more processors are individually or collectively programmed to further identify the at least one cancer methylation marker by processing methylation data of solid tumor samples, normal tissue samples, cell-free nucleic acid samples, or a combination thereof, obtained from one or more additional subjects.

47. The system of claim 46, wherein the at least one cancer methylation marker comprises epialleles, individual CpG sites, genomic regions, or a combination thereof.

48. The system of claim 46, wherein processing the methylation data comprises identifying the at least one cancer methylation marker based on a differential methylation of the at least one cancer methylation marker between the solid tumor samples, the normal tissue samples, the cell-free nucleic acid samples, or the combination thereof.

49. The system of claim 46, wherein the one or more additional subjects comprise one or more cancer patients and one or more normal subjects.

50. The system of claim 49, wherein processing the methylation data comprises identifying the at least one cancer methylation marker based on a differential methylation of the at least one cancer methylation marker between samples obtained from the one or more cancer patients and samples obtained from the one or more normal subjects.

51. A non-transitory computer-readable storage medium storing a set of instructions that, when executed, cause one or more processors to detect copy number variants (CNVs) from a plurality of cell-free nucleic acids of a subject, the set of instructions comprising instructions to:
obtain a plurality of sequencing reads derived by sequencing the plurality of cell-free nucleic acids, wherein the plurality of sequencing reads comprises (i) a plurality of tumor-derived sequencing reads corresponding to tumor-derived cell-free nucleic acids of the plurality of cell-free nucleic acids and (ii) a plurality of normal sequencing reads corresponding to normal cell-free nucleic acids of the plurality of cell-free nucleic acids; and
use methylation sequencing data of the plurality of cell-free nucleic acids and at least one cancer methylation marker to distinguish the plurality of tumor-derived sequencing reads from the plurality of normal sequencing reads, wherein distinguishing the plurality of tumor-derived sequencing reads from the plurality of normal sequencing reads comprises:
classifying a sequencing read of the methylation sequencing data as a tumor-derived sequencing read or a normal sequencing read;
constructing a profile of tumor-derived sequencing read counts, wherein constructing the profile comprises quantifying the plurality of tumor-derived sequencing reads at each of a plurality of genomic regions;
normalizing the constructed profile of tumor-derived sequencing read counts, to produce a normalized profile of tumor-derived sequencing read counts; and
inferring a CNV status for each of the plurality of genomic regions based on the normalized profile of tumor-derived sequencing read counts.

52. The non-transitory computer-readable storage medium of claim 51, wherein classifying a sequencing read of the methylation sequencing data as a tumor-derived sequencing read or a normal sequencing read comprises at least one of:
(i) calculating a likelihood ratio for the sequencing read, and comparing the likelihood ratio to a likelihood ratio threshold, wherein a likelihood ratio that exceeds the likelihood ratio threshold indicates a tumor-derived sequencing read; and
(ii) calculating a posterior probability for the sequencing read, and comparing the posterior probability to a posterior probability threshold, wherein a posterior probability that exceeds the posterior probability threshold indicates a tumor-derived sequencing read.

53. The non-transitory computer-readable storage medium of claim 51 or 52, wherein classifying the sequencing read as a tumor-derived sequencing read or a normal sequencing read further comprises:
calculating a class-specific likelihood for the sequencing read.

54. The non-transitory computer-readable storage medium of any of claims 51-53, wherein constructing the profile of tumor-derived sequencing read counts comprises excluding all of the plurality of sequencing reads classified as a normal sequencing read.

55. The non-transitory computer-readable storage medium of any of claims 51-53, wherein constructing the profile of tumor-derived sequencing read counts comprises dividing at least a portion of the human genome into the plurality of genomic regions, the plurality of genomic regions comprising non-overlapping bins, according to a genome-wide segmentation strategy.

56. The non-transitory computer-readable storage medium of claim 55, wherein the non-overlapping bins have a fixed size.

57. The non-transitory computer-readable storage medium of claim 55, wherein the non-overlapping bins vary in size.

58. The non-transitory computer-readable storage medium of any of claims 51-57, wherein normalizing the constructed profile of the tumor-derived sequencing read counts comprises calculating a fraction of tumor-derived cell-free nucleic acids in each of the plurality of genomic regions of the constructed profile.

59. The non-transitory computer-readable storage medium of any of claims 51-58, wherein normalizing the constructed profile of the tumor-derived sequencing read counts comprises performing a bias correction of the constructed profile.

60. The non-transitory computer-readable storage medium of claim 59, wherein performing the bias correction reduces bias attributable to at least one of: GC contents, sequencing read mapping, sequencing library construction, and sequencing platforms.

61. The non-transitory computer-readable storage medium of claim 59, wherein performing the bias correction comprises comparing the constructed profile to a reference profile.

62. The non-transitory computer-readable storage medium of claim 61, wherein the reference profile is a matched normal sample comprising genomic DNA from white blood cells obtained from a same blood sample as the plurality of cell-free nucleic acids.

63. The non-transitory computer-readable storage medium of claim 61, wherein the reference profile is constructed from one or more cfDNA samples obtained from healthy subjects.

64. The non-transitory computer-readable storage medium of claim 61, wherein the reference profile is constructed from certain genomic regions within a same sample.

65. The non-transitory computer-readable storage medium of any of claims 51-64, wherein normalizing the constructed profile of tumor-derived sequencing read counts comprises measuring log ratios between case and control samples for each of the plurality of genomic regions.

66. The non-transitory computer-readable storage medium of any of claims 51-65, wherein the set of instructions comprises instructions to detect a cancer of the subject based on the plurality of inferred CNV statuses.

67. The non-transitory computer-readable storage medium of claim 66, wherein the cancer is detected based on a fraction of one or more genomic regions having tumor-derived sequencing read counts, and wherein the detecting comprises using a fraction of the plurality of genomic regions having abnormal sequencing read counts as a cancer indicator score, wherein a genomic region is determined to have an abnormal sequencing read count based on a log ratio of the inferred CNV status of the genomic region.

68. The non-transitory computer-readable storage medium of any of claims 51-67, wherein the set of instructions comprises instructions to use the CNV status for treatment monitoring of the subject.

69. The non-transitory computer-readable storage medium of any of claims 51-67, wherein the set of instructions comprises instructions to use the CNV status for patient stratification of the subject.

70. The non-transitory computer-readable storage medium of any of claims 51-67, wherein the set of instructions comprises instructions to use the CNV status for tracing a tissue-of-origin of the plurality of cell-free nucleic acids.

71. The non-transitory computer-readable storage medium of any of claims 51-67, wherein the set of instructions comprises instructions to identify the at least one cancer methylation marker by processing methylation data of solid tumor samples, normal tissue samples, cell-free nucleic acid samples, or a combination thereof, obtained from one or more additional subjects.

72. The non-transitory computer-readable storage medium of claim 71, wherein the at least one cancer methylation marker comprises epialleles, individual CpG sites, genomic regions, or a combination thereof.

73. The non-transitory computer-readable storage medium of claim 71, wherein processing the methylation data comprises identifying the at least one cancer methylation marker based on a differential methylation of the at least one cancer methylation marker between the solid tumor samples, the normal tissue samples, the cell-free nucleic acid samples, or the combination thereof.

74. The non-transitory computer-readable storage medium of claim 71, wherein the one or more additional subjects comprise one or more cancer patients and one or more normal subjects.

75. The non-transitory computer-readable storage medium of claim 74, wherein processing the methylation data comprises identifying the at least one cancer methylation marker based on a differential methylation of the at least one cancer methylation marker between samples obtained from the one or more cancer patients and samples obtained from the one or more normal subjects.

76. A method for detecting fetal copy number variants (CNVs) from a plurality of cell-free nucleic acids of a maternal sample of a pregnant subject, the method comprising:
obtaining a plurality of sequencing reads derived by sequencing the plurality of cell-free nucleic acids, wherein the plurality of sequencing reads comprises (i) a plurality of fetal-derived sequencing reads corresponding to fetal-derived cell-free nucleic acids of the plurality of cell-free nucleic acids and (ii) a plurality of normal sequencing reads corresponding to normal cell-free nucleic acids of the plurality of cell-free nucleic acids;
using methylation sequencing data of the plurality of cell-free nucleic acids and at least one fetal methylation marker to distinguish the plurality of fetal-derived sequencing reads from the plurality of normal sequencing reads, wherein distinguishing the plurality of fetal-derived sequencing reads from the plurality of normal sequencing reads comprises:
classifying a sequencing read of the methylation sequencing data as a fetal-derived sequencing read or a normal sequencing read;
constructing a profile of fetal-derived sequencing read counts, wherein constructing the profile comprises quantifying the plurality of fetal-derived sequencing reads at each of a plurality of genomic regions;
normalizing the constructed profile of fetal-derived sequencing read counts, to produce a normalized profile of fetal-derived sequencing read counts; and
inferring a CNV status for each of the plurality of genomic regions based on the normalized profile of fetal-derived sequencing read counts.

77. The method of claim 76, wherein classifying a sequencing read of the methylation sequencing data as a fetal-derived sequencing read or a normal sequencing read comprises at least one of:
(i) calculating a likelihood ratio for the sequencing read, and comparing the likelihood ratio to a likelihood ratio threshold, wherein a likelihood ratio that exceeds the likelihood ratio threshold indicates a fetal-derived sequencing read; and
(ii) calculating a posterior probability for the sequencing read, and comparing the posterior probability to a posterior probability threshold, wherein a posterior probability that exceeds the posterior probability threshold indicates a fetal-derived sequencing read.

78. The method of claim 76 or 77, wherein classifying the sequencing read as a fetal-derived sequencing read or a normal sequencing read further comprises: calculating a class-specific likelihood for the sequencing read.

79. The method of any of claims 76-78, further comprising using the CNV status to identify a fetus of the pregnant subject as having or being suspected of having a disease or disorder.

80. The method of claim 79, wherein the disease or disorder is a fetal aneuploidy.

81. The method of claim 80, wherein the fetal aneuploidy is Down Syndrome.

82. The method of any of claims 76-81, wherein constructing the profile of fetal-derived sequencing read counts comprises dividing at least a portion of the human genome into the plurality of genomic regions, the plurality of genomic regions comprising non-overlapping bins, according to a genome-wide segmentation strategy.

83. The method of claim 82, wherein the non-overlapping bins have a fixed size.

84. The method of claim 82, wherein the non-overlapping bins vary in size.

85. The method of claim 82, wherein normalizing the constructed profile of the fetal-derived sequencing read counts comprises calculating a fraction of fetal-derived cell-free nucleic acids in each of the plurality of genomic regions of the constructed profile.
